(19) United States
(12) Patent Application Publication
(10) Pub. No.: US 2021/0110888 A1
(43) Pub. Date: Apr. 15, 2021

(54) METHOD AND SYSTEM FOR ASSIGNING RISK FACTORS TO INDIVIDUALS

(71) Applicant: MOUNT SINAI GENOMICS, INC., Stamford, CT (US)

(72) Inventors: Lisa J. Edelmann, Bronx, NY (US); Ashley Helena Birch, Stamford, CT (US); Moara Machado, Stamford, CT (US)

(21) Appl. No.: 17/067,300

(22) Filed: Oct. 9, 2020

Related U.S. Application Data
(60) Provisional application No. 62/913,876, filed on Oct. 11, 2019.

(51) Int. Cl. G16B 30/00; C12Q 1/6869; G16H 10/40; G16H 10/60; G16H 50/30; G16H 70/60

(52) U.S. Cl. CPC G16B 30/00; C12Q 1/6869; G16H 70/60; G16H 10/60; G16H 50/30; G16H 10/40

(57) ABSTRACT

The present disclosure relates to a method that may include retrieving an individual profile for an individual and a sequence dataset associated with the individual profile. The method may include determining an ancestral composition of the sequence dataset. The ancestral composition includes one or more ancestral groups. The method may also include retrieving one or more group residual risk values corresponding to the one or more ancestral groups. Each group residual risk value may be specific to an ancestral group and determined based on a carrier frequency and a detection rate specific to the ancestral group. The method may also include assigning metadata to the individual profile. The metadata may include a personalized residual risk of the individual. The personalized residual risk may be determined based on the one or more group residual risk values.

### CROSS REFERENCE TO RELATED APPLICATION

This application claims the benefit of U.S. Provisional Patent Application No. 62/913,876, filed on Oc. 11, 2019, which is hereby incorporated by reference in its entirety.

### FIELD

The present invention relates to a method for assigning metadata to an individual profile and, more specifically, to determining the metadata based on sequence data.

### BACKGROUND

Genetic testing is becoming increasingly common. Individuals have genetic testing for a variety of reasons. In some situations, as with adoptions, in vitro fertilization, and surrogate motherhood, the offspring could have a desire or need to locate the biological parents. Other individuals have a medical interest in genetic testing for screening to determine whether they are a carrier for a genetic trait or disease, the likelihood they will exhibit the trait or disease, or the risk that their offspring will be a carrier or exhibit the trait or disease. Other reasons for testing involve forensic genetics for providing information and evidence to solve crimes.

Different ancestral traits and their affiliation to diseases can help scientists to determine appropriate approaches of treatment. Human genetics deals with three types of DNA; autosomal DNA, X or Y sex chromosome DNA, or mitochondrial DNA. Autosomal DNA is a term used in genetic genealogy to describe DNA which is inherited from the autosomal chromosomes. An autosome is any of the numbered chromosomes, as opposed to the sex chromosomes. Humans have 22 pairs of autosomes and one pair of sex chromosomes, e.g. the X chromosome and the Y chromosome, such as the XY combination that defines a male and the XX combination that defines a female. Mitochondrial DNA is the small circular chromosome found inside mitochondria. Mitochondrial DNA is passed almost exclusively from mother to offspring through the egg cell.

With advances in genetic testing, it has become possible to test for the presence of pathogenic variants causing autosomal or X-linked recessive disorders, which can cause disease when passed down to future offspring. Accurate risk assessment is beneficial for reproductive couples known to have certain diseases in their families or to quantify the risk of offspring exhibiting a disease unbeknownst to the parents due to one or both parents being carriers.

### SUMMARY

Prior methods for carrier screening and risk assessment have relied upon genetic carrier frequency information and whether an individual is a carrier for one or more causal genetic variants of interest. However, errors such as false negatives are commonly associated with such information. For example, a false positive may occur where an individual is incorrectly reported to be a carrier. It is also possible that the individual is determined to have a low carrier risk when in actuality the individual has a higher carrier frequency and risk than is reported due to having a different ethnicity than what it is thought, or is a carrier despite the test indicating a negative result.

Attempts have been made to remove the subjectivity or errors associated with self-reported ancestry by using ancestry informative markers (AIMs). These AIMs are generally single-nucleotide polymorphisms, e.g. a modification of a single nucleotide base within a DNA sequence, that are exhibited in substantially different frequencies amongst different populations. The limitation of using an AIM is that, at most, it provides a potential means to check the genotyping of a sample against a particular mutation, such as a founder mutation or variant, which is a genetic alteration observed with high frequency in a group that is or was geographically or culturally isolatedwhere one or more of the ancestors was a carrier of the altered gene. However, AIMs are not useful for providing a personal residual risk assessment, particularly across a large range of pathogenic genetic variants in various regions of the genetic code because they provide limited information regarding an indivdival's full ancestry and are mainly used as a confirmatory method to genotyping for founder alleles.

Described herein are methods for utilizing low-pass sequencing to determine global ancestry of individual samples to accurately identify the ancestral background of the individual. The result from low-pass sequencing is used in conjunction with user residual risks based on carrier frequencies and detection rates that are specific for each ethnic group. The method provides a personalized residual risk that is informed by the individual's global molecular ancestral makeup. Unique and accurate individual carrier screen results are provided. These results can be used to provide a personalized residual risk assessment for the individual, the probability of a reproductive couple having an offspring with a certain genetic disease, and more complete and accurate information for a reproductive couple when evaluating reproductive options with genetic counselors and health care professionals.

In some embodiments, systems and methods for assigning data to a dataset are described. In some embodiments, a method may include retrieving an individual profile for an individual and a sequence dataset associated with the individual profile. The method may also include determining an ancestral composition of the sequence dataset, the ancestral composition comprising one or more ancestral groups. The method may further include retrieving one or more group residual risk values corresponding to the one or more ancestral groups, each group residual risk value specific to an ancestral group and determined based on a carrier frequency and a detection rate specific to the ancestral group. The method may further include assigning metadata to the individual profile, the metadata comprising a personalized residual risk of the individual, the personalized residual risk determined based on the one or more group residual risk values.

These and other aspects of the present invention will become apparent from the disclosure herein.

### BRIEF DESCRIPTION OF THE DRAWINGS

FIG. 1 illustrates a diagram of a system environment of an example computing system, in accordance with some embodiments.

FIG. 2 is a flowchart depicting an example process for performing a carrier risk assessment process for an individual, in accordance with some embodiments.

FIG. 3 is a flowchart depicting an example expanded carrier screening process, in accordance with some embodiments.

FIG. 4 is a flowchart depicting an example residual risk determination process, in accordance with some embodiments.

FIG. 5 illustrates an example of the classification of ancestral groups that are formed by binning one or more ethnicities into an ancestral group, in accordance with some embodiments.

FIG. 6 is a block diagram illustrating components of an example computing machine, in accordance with some embodiments.

The figures depict various embodiments for purposes of illustration only. One skilled in the art will readily recognize from the following discussion that alternative embodiments of the structures and methods illustrated herein may be employed without departing from the principles described herein.

### DETAILED DESCRIPTION

The figures (FIGS.) and the following description relate to preferred embodiments by way of illustration only. One of skill in the art may recognize alternative embodiments of the structures and methods disclosed herein as viable alternatives that may be employed without departing from the principles of what is disclosed.

Reference will now be made in detail to several embodiments, examples of which are illustrated in the accompanying figures. It is noted that wherever practicable similar or like reference numbers may be used in the figures and may indicate similar or like functionality. The figures depict embodiments of the disclosed system (or method) for purposes of illustration only. One skilled in the art will readily recognize from the following description that alternative embodiments of the structures and methods illustrated herein may be employed without departing from the principles described herein.

### DEFINITIONS

The term ancestry informative marker (“AIM”), as used herein, means a single-nucleotide polymorphism (SNP), e.g. a modification of a single nucleotide base within a DNA sequence.

The term “Bayesian” as used herein means the use of Bayesian statistical methods using Bayes' theorem to compute probabilities.

The term biomarker may include a suitable nucleic acid marker, such as a SNP, a genotype, a haplotype, an allele, or a non-nucleic acid marker, such as a protein sequence, a phenotype, etc.

The term causal genetic variants (“CGVs”) means disease-causing alleles or variants found in a human or animal population which manifest a given disease.

The term “ethnicity” refers to a group or population of individuals who are defined by a common genealogy.

The term “founder mutation” means: a genetic alteration observed with high frequency in a group that is or was geographically or culturally isolated, in which one or more of the ancestors was a carrier of the altered gene. This phenomenon is often called a founder effect. It is called the founder variant.

The term “individual” refers to a human individual, living or non-living. For example, an individual could be a prospective offspring of a reproductive couple.

The term “molecular ancestry” means the genealogical lineage as determined or traced by various genetic markers or traits. The term “genetic ancestry” can be used as an alternative to molecular ancestry. Molecular ancestry or genetic ancestry can be determined on a global or local basis. A global basis may refer to the average of the molecular ancestry percentages across the 23 chromosome pairs. A local basis may describe the ethnic origin of a DNA segment that contains a specific gene and includes a haplotype that can be identified as belonging to a specific ethnic group.

The term “patient” or “subject” means an individual who would be a candidate for the tests, methods and products described herein.

The term “reproductive couple” means a pair of individuals who can potentially produce offspring through sexual intercourse, assisted reproductive technology, or other methods, including e.g., artificial insemination or in vitro fertilization. The reproductive couple would include a female member (a reproductive female or prospective mother) and a male member (a reproductive male or prospective father). The term “reproductive couple” can be used as an alternative to the term “prospective parents”, comprising a” prospective mother” and a “prospective father”.

The “residual risk”, also abbreviated “RR”, has a general definition of the amount of risk or danger associated with an action or event remaining after natural or inherent risks have been reduced by risk controls. In this disclosure, the term “residual risk” may refer to the probability that an individual (or his/her offspring) is still a carrier of a genetic disease or has the genetic disease after a negative result of genetic screening of the genetic disease.

The terms “sequence information” and “genotyping information” are both used to describe the genetic nucleotide information or sequences determined from a DNA or RNA polynucleotide sample.

### EXAMPLE SYSTEM ENVIRONMENT

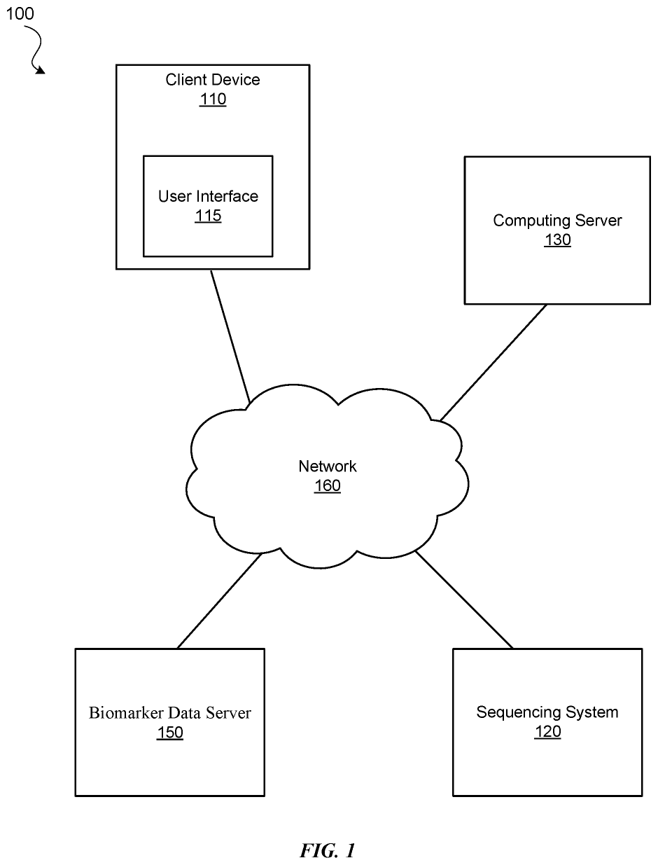

FIG. 1 illustrates a diagram of a system environment 100 of an example computing system, in accordance with some embodiments. The system environment 100 shown in FIG. 1 includes a client device 110, a sequencing system 120, a computing server 130, a biomarker data server 150, and a network 160. In various embodiments, the system environment 100 may include fewer or additional components. The system environment 100 may also include different components. While some of the components in the system environment 100 may at times be described in a singular form while other components may be described in a plural form, the system environment 100 may include one or more of each of the components. For simplicity, multiple instances of a type of entity or component in the system environment 100 may be referred to in a singular form even though the system may include one or more such entities or components. For example, in one embodiment, while the client device 110 may be referred to in a singular form, a computing server 130 may serve multiple customers, each being associated with a client device 110. Likewise, the computing server 130 may rely on multiple biomarker data servers 150. Conversely, a component described in the plural form does not necessarily imply that more than one copy of the component is always needed in the environment 100.

The client device 110 is a computing device capable of communicating to the computing server 130 via a network 160. Examples of computing devices include desktop computers, laptop computers, personal digital assistants (PDAs), smartphones, tablets, wearable electronic devices (e.g., smartwatches), smart household appliance (e.g., smart televisions, smart speakers, smart home hubs), Internet of Things (IoT) devices or other suitable electronic devices. In one embodiment, a client device 110 executes an application that launches a graphical user interface (GUI) for a user of the client device 110 to interact with the computing server 130. The GUI may be an example of a user interface 115. For example, a client device 110 may execute a web browser application such as a web form to enable interactions between the client device 110 and the computing server 130 via the network 160. In some embodiments, the user interface 115 may take the form of a software application published by the computing server 130 and installed on the user device 110. In some embodiments, a client device 110 interacts with the computing server 130 through an application programming interface (API). The user interface 115 may receive data and results from the computing server 130 and display the results.

The sequencing system 120 may include various sequencing machines to extract genetic data from biological samples (e.g., saliva, blood, hairs, tissues) of individuals, who may be referred to as subjects or patients. The sequencing system 120 may use various nucleotide processing techniques such as amplification and sequencing. Amplification may include using polymerase chain reaction (PCR) to amplify segments of nucleotide samples. Sequencing may include deoxyribonucleic acid (DNA) sequencing, ribonucleic acid (RNA) sequencing, etc. Suitable sequencing techniques may include Sanger sequencing and massively parallel sequencing such as various next-generation sequencing (NGS) techniques including whole genome sequencing, low-pass whole genome sequencing, pyrosequencing, sequencing by synthesis, sequencing by ligation, and ion semiconductor sequencing. For simplicity, various massively parallel sequencing techniques may be referred collectively as NGS techniques. The sequencing system 120 performs sequencing of the biological samples and determines the nucleotide sequences of the individuals. The sequencing system 120 generates data of the sequences of individuals' genome or part of the genome based on the sequencing results. The data may include data sequenced from DNA or RNA and may include base pairs from coding and/or non-coding regions of the genome. The sequence datasets may be provided to computing server 130 for further processing and analyses.

The sequencing system 120 may perform various steps in preparing a nucleic acid sample for NGS sequencing, in accordance with some embodiments. The sequencing system 120 extracts a nucleic acid sample (DNA or RNA) from a biological sample of a subject. The sample can be any subset of the human genome or the whole genome. The biological sample can include blood, plasma, serum, urine, fecal, saliva, other types of bodily fluids, or any combination thereof. In some embodiments, methods for drawing a blood sample (e.g., syringe or finger prick) can be less invasive than procedures for obtaining a tissue biopsy, which can require surgery.

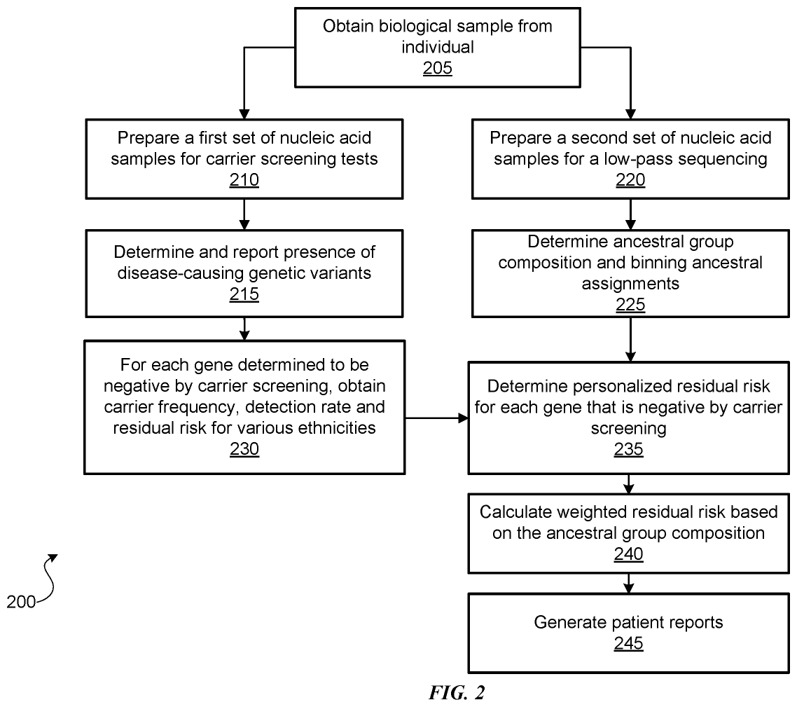

The sequencing system 120 prepares a sequencing library from the biological sample. The sequencing library may include multiple sets of nucleic acid samples. For example, for reasons that will be discussed in further detail below with reference to FIG. 2, the sequencing system 120 may prepare a first set of nucleic acid samples for a high-resolution sequencing and a second set of nucleic acid samples for a low-pass sequencing.

During the library preparation for NGS, the nucleic acid samples are randomly cleaved into thousands or millions of fragments. Unique molecular identifiers (UMI) are added to the nucleic acid fragments (e.g., DNA fragments) through adapter ligation. The UMIs are short nucleic acid sequences (e.g., 4-10 base pairs) that are added to ends of DNA fragments during adapter ligation. In some embodiments, UMIs are degenerate base pairs that serve as a unique tag that can be used to identify sequence reads originating from a specific DNA fragment. During PCR amplification following adapter ligation, the UMIs are replicated along with the attached DNA fragment, which provides a way to identify sequence reads that came from the same original fragment in downstream analysis.

In sequencing, the sequencing system 120 generates sequence reads from the nucleic acid samples. Sequencing data can be acquired from the known sequencing techniques in the art. For example, the sequencing can include synthesis technology (ILLUMINA), pyrosequencing (454 LIFE SCIENCES), ion semiconductor technology (Ion Torrent sequencing), single-molecule real-time sequencing (PACIFIC BIOSCIENCES), sequencing by ligation (SOLiD sequencing), nanopore sequencing (OXFORD Nanopore Technologies), or paired-end sequencing. In some embodiments, massively parallel sequencing is performed using sequencing-by-synthesis with reversible dye terminators.

In some embodiments, the sequence reads can be aligned to a reference genome to determine the alignment position information. The alignment position information can indicate a beginning position and an end position of a region in the reference genome that corresponds to a beginning nucleotide base and end nucleotide base of a given sequence read. Alignment position information can also include sequence read length, which can be determined from the beginning position and end position. A region in the reference genome can be associated with a gene or a segment of a gene.

The sequencing system 120 may perform different types of sequencing such as Sanger sequencing and massively parallel sequencing for various purposes. The resolution for the sequencing may also be different, depending on the purpose. For example, in one case, a high-resolution sequencing may be performed to determine the variant (e.g., a SNP) at a specific genetic locus. In other cases that will be discussed below, a low-resolution sequencing (low-pass sequencing) may also be performed over largely the whole genome (or a large portion of the genome) of a subject.

The resolution of a sequencing (particularly in NGS) may be measured in terms of the coverage of the sequencing, which describes the average number of reads that align to known reference bases. A particular location may have a sequence depth (the number of reads at that location). Owing to the random cleavage nature of NGS, the depths at different genomic locations are random and often exhibit a distribution such as a Poisson distribution or a Gaussian distribution. A sequencing coverage of 20× may refer to a mean (or medium, depending on implementation) depth of 20 in the distribution. The coverage may also be expressed as an inter-quartile range such as a coverage of at least 10× between 25th and 75th percentiles of depths in various genomic locations.

A high-resolution sequencing may refer to a Sanger sequencing or an NGS sequencing that has a high coverage, usually 10× or higher. In some embodiments, a high-resolution sequencing has a sequencing coverage between 10× and 20×. In some embodiments, a high-resolution sequencing has a sequencing coverage between 20× and 30×. In some embodiments, a high-resolution sequencing has a sequencing coverage between 30× and 50×. In some embodiments, a high-resolution sequencing has a sequencing coverage between 50× and 100×. In some embodiments, a high-resolution sequencing has a sequencing coverage of over 100×.

A low-resolution sequencing (low-pass sequencing) may refer to sequencing that has a lower coverage, usually 5× or lower. In some embodiments, a low-pass sequencing has a sequencing coverage between 1× and 5×. In some embodiments, a low-pass sequencing has a sequencing coverage between 0.5 and 1×. In some embodiments, a low-pass sequencing has a sequencing coverage between 0.3× and 0.5×. In some embodiments, a low-pass sequencing has a sequencing coverage between 0.1× and 0.3×. A low-pass sequencing is often nosier but less expensive to run compared to a high-resolution sequencing. For a single run in an NGS sequencing machine, more subject samples can fit into the run if a low-pass sequencing is used. For example, the coverage of 0.4× may occupy only about 1% of the capacity of the run compared to the coverage of 40×. Despite a low average sequence depth, the covered location in the genome can be broad. For example, a low-pass sequencing may cover a large section or substantially the entire genome.

Other types of sequencing techniques may also be used, such as ligation-dependent probe amplification (MLPA), SNPlex from APPLIED BIOSYSTEMS (ABI), AGENA MALDI-TOF genotyping, LUMINEX, or suitable Sanger sequencing techniques. Some of those techniques may be used to determine a small number of SNPs (e.g., fewer than 100 SNPs). For arrays that cover a larger number of SNPs (e.g., hundreds of thousands or millions), AFFYMETRIX array, AGILENT SNP arrays, ILLUMINA INFINIUM may also be used.

The sequencing may be random sequencing or targeted sequencing. Random sequencing may include the use of NGS techniques that randomly sequence various locations in the genome. A target sequencing may use the data from a target NGS library (both on and off target sequences) or use other techniques such as various types of Sanger sequencing.

After sequencing, the sequencing system 120 may generate one or sequence datasets for a subject. The length of a sequence dataset may vary, depending on the type of sequencing techniques used. For example, in a Sanger sequencing, a run of the sequencing may generate a sequence dataset of 200-500 base pairs, although results from multiple runs at different genomic locations may also be combined to generate a single sequence dataset. For NGS, the length of a sequence dataset for a single run may typically be ranged from 0.1 Mbp (millions of base pairs) to 100 Mbp or even longer. In some embodiments, the length of the sequence dataset is in the order of magnitude of 1,000 base pairs. In some embodiments, the length of the sequence dataset is in the order of magnitude of 10,000 base pairs. In some embodiments, the length of the sequence dataset is in the order of magnitude of 10,000 base pairs. In some embodiments, the length of the sequence dataset is in the order of magnitude of 100,000 base pairs (0.1 Mbps). In some embodiments, the length of the sequence dataset is in the order of magnitude of 1 Mbp. In some embodiments, the length of the sequence dataset is in the order of magnitude of 10 Mbps. In some embodiments, the length of the sequence dataset is in the order of magnitude of 100 Mbps. In some embodiments, the length of the sequence dataset is in the order of magnitude that is greater than 100 Mbps.

An output file of the sequence data having SAM (sequence alignment map) format or BAM (binary) format may be generated and output for further analysis such as variant calling. A sequence dataset may sometimes also be referred to as a DNA dataset, a genetic dataset, a genotype dataset, a haplotype dataset, depending on the nature of the data in the sequencing dataset. The output file may be provided to the computing server 130 for further analysis.

The computing server 130 may include one or more computing devices that perform analysis of sequence data provided by the sequencing system 120. The computing server 130 may perform genetic and carrier screening for individuals, such as pre-conception screening for prospective parents to determine the risk of a prospective offspring having a genetic disease. The computing server 130 may also perform carrier screenings for other individuals, whether the individuals are planning to have children or not.

The computing server 130 may perform a carrier screening a genetic disease using a high-resolution sequencing to determine whether the subject has one or more pathogenic variants in a gene that is associated with the disease. Pathogenetic variants may also be referred to as CGVs. In response to a determination that the subject having one or more pathogenic variants, the computing server 130 may assign a risk factor of the subject carrying the disease based on one or more statistical models. The computing server 130 may screen for a list of genetic diseases. For example, the list may include more than 200 genetic diseases. Some or all of the diseases may be autosomal recessive or X-linked diseases. Typically, a subject is determined to be a carrier in a range of zero to 10 genetic diseases. The computing server 130 may return negative results for the rest of the genetic diseases in the list for the carrier screening.

For the rest of genetic diseases that have negative screen results, the computing server 130 may perform another sequencing analysis process to determine the residual risk of the subject being a carrier for those diseases. The computing server 130 may retrieve a sequencing dataset of the subject that is generated by a low-pass sequencing that has a low averaged sequencing depth but covers a large genomic region (such as a significant portion of the genome or the entire genome) of the subject. The computing server 130 may align the sequencing dataset to one or more reference genomes of different ethnicity origins provided by the biomarker data server 150. The computing server 130 may determine the molecular ancestral composition of the sequencing dataset. Based on the ancestral composition and the residual risk values of each ancestral group in the ancestral composition, the computing server 130 may determine a personalized residual risk of an individual associated with a particular disease. The residual risk may be the risk of a prospective parent being a carrier of the disease. The residual risk may also be the risk of a prospective offspring having the disease. Different diseases may have different residual risk values.

The computing server 130 may store a plurality of individual profiles associated with various individuals. An individual profile may be a profile for a user or a prospective offspring. An individual profile may include profile metadata such as name, date of birth, self-reported ethnicity, parent information, consented health information, and other information. An individual profile may also include metadata that is associated with the genetic screening and residual risk results of an individual. For example, the metadata may be saved as key-value pairs or in a tabular form. Upon determining the residual risk values of various diseases, the computing server 130 may assign the metadata to the individual profile. The computing server 130 may receive a request for a report related to the individual, such as a genetic screen report. The computing server 130 may retrieve the data and generate a report. The payload of the report may be sent via the network 160 to be displayed at the user interface 115 of the client device 110. The report may be a patient report such as a clinical report.

In various embodiments, the computing server 130 may take different forms. The computing server 130 may be a server computer that includes software and one or more processors to execute code instructions to perform various processes described herein. The computing server 130 may also be a pool of computing devices that may be located at the same geographical location (e.g., a server room) or be distributed geographically (e.g., cloud computing, distributed computing, or in a virtual server network). The computing server 130 may also provide an application programing interface (API) for various devices in the environment 100 to communicate with the organization computing server 130.

A biomarker data server 150 may be a data server that provides information regarding various biomarkers. One of the biomarker data servers 150 may be part of the computing server 130 and other biomarker data servers 150 may be third party databases or data providers. Suitable data servers may include genomic coordinate and sequence sources that may provide data regarding sequences of genomes for humans and other organisms, such as a reference library for human genomes of various ethnic origins. Various biomarker data servers 150 may also be a sequence version source that may provides data regarding different sequence versions in various genetic loci, a gene name source that may provide nomenclature of genes, a mutation data source that may provide data regarding common mutations, and variant-phenotype relation database that may provide data regarding the association among a phenotype and one or more genetic loci or single nucleotide polymorphism (SNP). Example biomarker data servers 150 may include the University of California, Santa Cruz (UC SC) Genome Browser, the HUGO Gene Nomenclature Committee (HGNC; via genenames.org), the European Bioinformatics Institute and the Wellcome Trust Sanger Institute Ensembl Genome Browser, National Center for Biotechnology Information (NCBI) ClinVar, and the Qiagen Human Gene Mutation Database (HGMD). Other biomarker data servers 150 may include databases that store clinical study data, scientific papers, medical records, and suitable university databases.

The communications between the client devices 110, the sequencing system 120, the computing server 130, the biomarker data server 150 may be transmitted via a network 160, for example, via the Internet. The network 160 provides connections to the components of the system 100 through one or more sub-networks, which may include any combination of local area and/or wide area networks, using both wired and/or wireless communication systems. In one embodiment, a network 160 uses standard communications technologies and/or protocols. For example, a network 160 may include communication links using technologies such as Ethernet, 802.11, worldwide interoperability for microwave access (WiMAX), 3G, 4G, Long Term Evolution (LTE), 5G, code division multiple access (CDMA), digital subscriber line (DSL), etc. Examples of network protocols used for communicating via the network 160 include multiprotocol label switching (MPLS), transmission control protocol/Internet protocol (TCP/IP), hypertext transport protocol (HTTP), simple mail transfer protocol (SMTP), and file transfer protocol (FTP). Data exchanged over a network 160 may be represented using any suitable format, such as hypertext markup language (HTML), extensible markup language (XML), or JSON. In some embodiments, all or some of the communication links of a network 160 may be encrypted using any suitable technique or techniques such as secure sockets layer (SSL), transport layer security (TLS), virtual private networks (VPNs), Internet Protocol security (IPsec), etc. The network 160 also includes links and packet switching networks such as the Internet.

Various components in FIG. 1 may have different relationships. For example, in some embodiments, the computing server 130 and sequencing system 120 may be operated by the same entity. In some embodiments, the system environment 100 may include multiple sequencing systems 120, which may be vendors of the operator of the computing server 130 that is in contractual relationships with the sequencing systems 120. In some embodiments, a medical practitioner or an end-user individual may ask a sequencing system 120 to generate a sequence dataset of the individual and the medical practitioner or the individual may upload the sequence dataset to the computing server 130 for further analyses.

### EXAMPLE CARRIER RISK ASSESSMENT PROCESS

FIG. 2 is a flowchart depicting an example process for performing a carrier risk assessment process 200 for an individual, in accordance with some embodiments. The expanded carrier screening process 200 may include a first round of genetic screening for multiple genetic diseases, such as autosomal recessive diseases or X-chromosome linked diseases. The result of the screening for a particular disease may include a positive result, which indicates that the individual is a carrier or has a statistically significant likelihood that the offspring may have the disease. A negative result indicates that there is no evidence that the individual is a carrier of the disease. The carrier risk assessment process 200 may also include a second round of analysis that determines, for the diseases that have negative results, the personalized residual risk values of the individual being a carrier of the diseases.

In some embodiments, a biological sample from an individual is obtained 205. The biological sample may be any suitable sample such as blood, plasma, serum, urine, fecal, saliva, other types of bodily fluids, or any combination thereof. The biological sample may be collected at a clinic or directly from the individual. Nucleic acid samples such as DNA samples may be extracted from the biological sample at a laboratory.

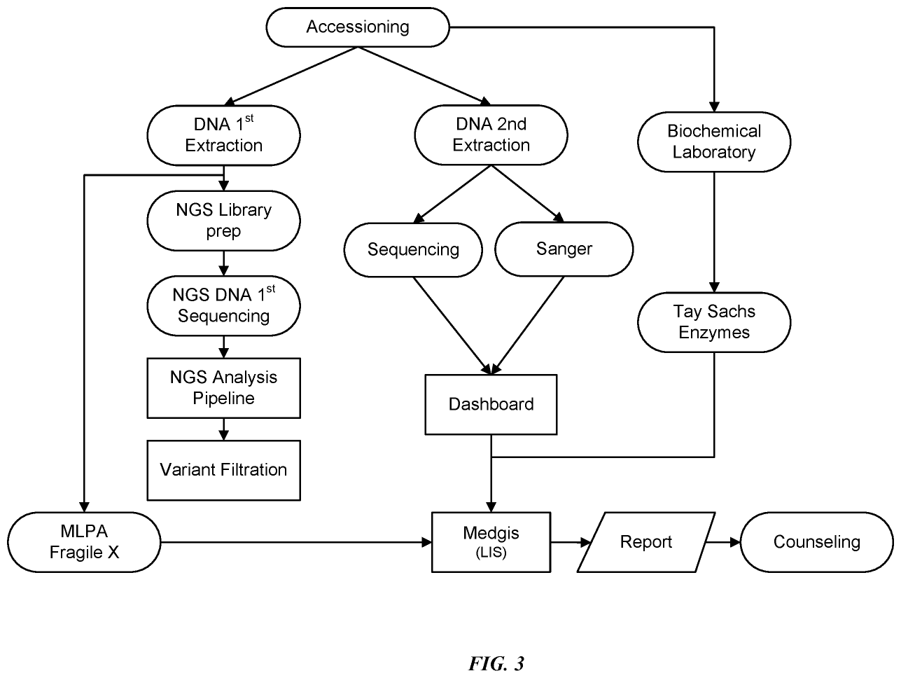

A first set of nucleic acid samples is prepared 210 from the biological sample. The first set of nucleic acid samples may be sub-divided into additional sets for various carrier screening tests. Collectively, those screen tests may be referred to as an extended carrier screening process 300, which will be discussed in further detail with reference to FIG. 3. One of the carrier screening tests may include a first sequencing on the first set of nucleic acid samples. The sequencing may include a high-resolution sequencing that determines whether the individual has one or more pathogenic variants related to one or more genetic diseases. For example, the high-resolution sequencing may be a high coverage (e.g., higher than 15×) NGS or a series of Sanger sequencing on certain targeted genetic loci.

Based on the carrier screening process, the presence of disease-causing genetic variants (pathogenic variants) is determined and reported 215. The extended carrier screening process 300 may screen for a list of pathogenic variants for various diseases. Typically, an individual may test positive for some of the pathogenic variants but extremely rarely an individual will test positive for all pathogenic variants. For some of the genetic diseases, the carrier screening may determine that the individual has a negative result. For each of the diseases for which there is a negative test result, a personalized residual risk that the individual will still be a carrier despite the negative result may be determined by analyzing the second set of nucleic acid samples.

From the biological sample, a second set of nucleic acid samples is prepared 220. In response to the negative result of one or more genetic diseases, a second sequencing on the second set of nucleic acid samples may be performed. The second sequencing may be a low-pass sequencing such as a low-pass whole genome sequencing (LPWGS). The LPWGS may start with the second set of nucleic acid samples that include the individual's entire (a significant portion) chromosomal DNA and DNA contained in the mitochondria. The LPWGS may have a coverage of about 0.4× to 5×. The range may be also be narrower as discussed with reference to sequencing system 120. While almost the entire genome is eligible for sequencing, due to the low coverage only about half of the genomic locations are sequenced. The read for most of the genomic locations can be low, such as having a depth of 1 or 2. Because the nucleic acid samples are randomly cleaved and selected in the sequencing, each run of low-pass sequencing may sequence different genomic locations. The genomic locations that are sequenced for two individuals may also be different. While low-pass sequencing is discussed in association with an example for performing the second sequencing, a high-resolution sequencing such as a regular whole genome sequencing may also be used for the second sequencing, although generally it is more expensive to perform a high-resolution sequencing. The second sequencing may also be a high resolution sequencing. Also, targeted sequencing may be used for global ancestry determination. For example, global ancestry may be determined from datat of targeted sequencing using on and off target data.

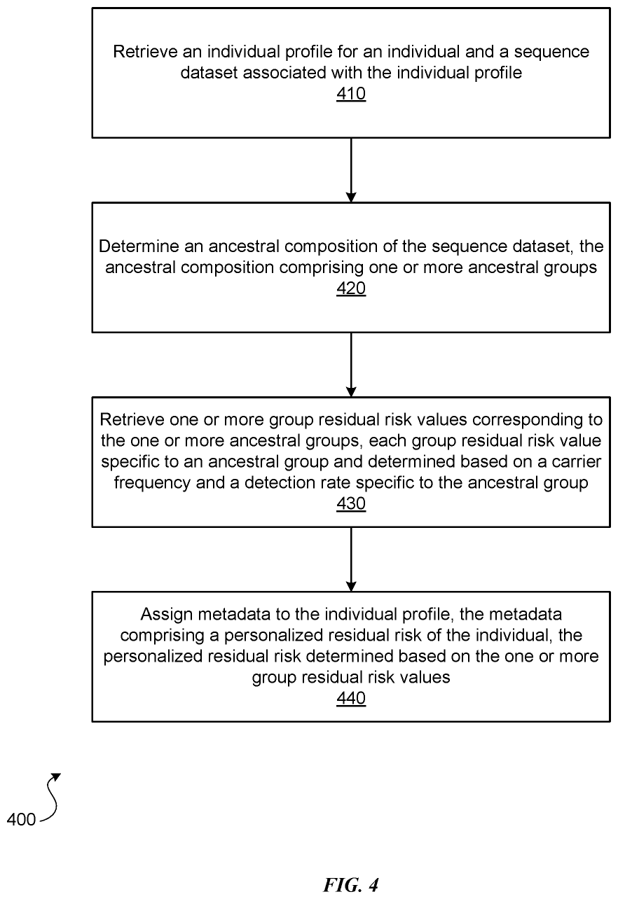

For the second set of nucleic acid samples, the ancestral group composition of the individual as reflected in the second set of nucleic acid samples is determined 225. The result of the second sequencing may be mapped and aligned to reference ancestry-specific genomes. The reference library may be retrieved from a biomarker data server 150. The ancestry determination is performed by utilizing a library of reference single nucleotide polymorphisms (SNPs). First, the sequence data obtained from LPWGS is aligned against the reference set. Once aligned, base calling is performed to identify any SNPs present in the sequencing data. After base calling, the identified SNPs are used to perform global ancestry analysis that assigns the global ancestry of the individual. The determination of an ancestral group composition and personalized residual risk will be discussed in further detail below with reference to FIG. 4.

Based on the variants that are determined negative in step 215 by carrier screening, the carrier frequency, detection rate and analytical detection rate are obtained 230 for each of the ancestral groups in the composition of the individual. Each ancestral group has a specific carrier frequency for a particular disease, which may also be referred to as the a priori risk of an individual belonging to the ancestral group to be a carrier of the disease.

A personalized residual risk is determined 235 for each gene that is negative by carrier screening. A weighted residual risk that is based on the fractional ancestral group composition may also be calculated 240. For example, each ancestral group may be associated with a group residual risk specific to a genetic disease. The weighted residual risk may be determined based on a weighted average of one or more group residual risk values weighted according to the molecular ancestral composition of the individual. A patient report may be generated 245 and be displayed at a graphical user interface 115.

In addition to determining the residual carrier risk of an individual, the carrier risk assessment process 200 may also be used to determine the risk of a prospective offspring of a reproductive couple having a genetic disease in the case where both parents are tested negative or one of the parents is tested negative. For example, the carrier risk assessment process 200 can be repeated for a second parent. The risk of the prospective offspring can be determined based on the combination of the residual risk or detected risk of the two prospective parents.

### EXAMPLE EXPANDED CARRIER SCREENING PROCESS

FIG. 3 is a flowchart depicting an example expanded carrier screening process 300, in accordance with some embodiments. The expanded carrier screening process 300 may correspond to step 215 in the process 200. The set of nucleic acid samples that are used for carrier screening tests may be further partitioned into two extractions. The first extraction is subject to NGS. NGS may be used as a tool to identify the presence of causal genetic variants (CGVs) corresponding to the individual being a carrier for a disease. A second extraction is used to perform genotyping and Sanger sequencing for variant confirmation that provides confirmation of the NGS calls (e.g., 25%) that are insertions/deletions, low quality, homozygous or mosaic, or in poor mapping regions.

Furthermore, the Sanger sequencing is used for sequencing of exons that do not meet 20× coverage across >99% of the exon and can be used to identify naming errors from NGS. Alongside NGS and sanger sequencing, various other methods may be applied in a disease-dependent basis.

For certain genes that are not amenable to sequencing genotyping, capillary electrophoresis or multiplex ligation-dependent probe amplification (MLPA) is used. Genotyping may be used for exon 10 of the cystic fibrosis gene (CFTR), while NGS may be used for other exons in CFTR. Owing to the challenges of sequencing this exon, relying solely upon NGS technology for testing the CFTR gene more likely will lead to false-positive results.

Capillary electrophoresis is used to estimate the number of CGG repeats in the FMR1 gene for Fragile X, which cannot be accurately performed using NGS technology. NGS is also used to identify non-repeat mutations to ensure the highest possible detection rates. In addition, samples with an intermediate result or larger (>45 CGG repeats) are reflexed to Southern blot to confirm repeat number & determine methylation status. Furthermore, AGG interruption reflex testing can be performed for premutation carriers to help quantify the likelihood of repeat expansion.

Multiplex ligation-dependent probe amplification (MLPA) is used to detect copy number changes in genes for which large deletions and duplications are common causes for diseases. Over 90% of pathogenic variants in HBA1/HBA2 (alpha-thalassemia) & SMN1(SMA-95-98%) are large deletions, thus MPLA may be employed for these genes. MLPA may also be employed for Duchenne/Becker muscular dystrophies for which about 60-70% pathogenic variants are large deletions or duplications in the DMD gene. To improve the detection rates, full gene sequencing may also be performed for the DMD gene to identify the additional 30-40% of pathogenic variants causative of DMD/BMD.

Although Tay-Sachs disease is more prevalent among Ashkenazi Jewish individuals, people of other ethnicities can also be carriers. DNA-only screening for the HEXA gene for Tay-Sachs can miss about 10% of carriers. Therefore, a combination of molecular and enzyme testing may be used for the most sensitive results. Enzyme testing for Tay-Sachs disease measures the level of Hex-A (Hexosaminidase A) in the blood with a high detection rate, regardless of the patient's ethnic background.

Shown in Table 1 below is a representative, non-limiting, list of the diseases that can be tested for in the expanded carrier screen. The genes controlling these diseases is indicated. A disease-causing variant in the gene would be considered a causal genetic variant. One of ordinary skill in the art would appreciate that this list can be expanded to include additional diseases, whether currently known or not yet known. The abbreviation “AR” means autosomal recessive and the abbreviation “XL” mean X chromosome-linked.

TABLE 1

| Gene | Inheritance | Disease name |
|---|---|---|
| ACADSB | AR | 2-methylbutyrylglycinuria |
| HSD3B2 | AR | 3-beta-hydroxysteroid dehydrogenase type II deficiency |
| MCCC1 | AR | 3-methylcrotonyl-CoA carboxylase deficiency (MCCC1-related) |
| MCCC2 | AR | 3-methylcrotonyl-CoA carboxylase deficiency (MCCC2-related) |
| OPA3 | AR | 3-methylglutaconic aciduria, type III |
| PHGDH | AR | 3-phosphoglycerate dehydrogenase deficiency |
| PTS | AR | 6-pyruvoyl-tetrahydropterin synthase deficiency |
| MTTP | AR | abetalipoproteinemia |
| AAAS | AR | achalasia-addisonianism-alacrimia syndrome |
| CNGA3 | AR | achromatopsia (CNGA3-related) |
| CNGB3 | AR | achromatopsia/progressive cone dystrophy |
| SLC39A4 | AR | acrodermatitis enteropathica |
| TRMU | AR | acute infantile liver failure |
| ACOX1 | AR | acyl-CoA oxidase I deficiency |
| EOGT | AR | Adams-Oliver syndrome 4 |
| ADA | AR | adenosine deaminase deficiency |
| TBX19 | AR | adrenocorticotropic hormone deficiency |
| ABCD1 | XL | adrenoleukodystrophy, X-linked |
| BTK | XL | agammaglobulinemia (X-linked) |
| FRMD4A | AR | agenesis of the corpus callosum |
| RNASEH2C | AR | Aicardi-Goutieres syndrome (RNASEH2C-related) |
| SAMHD1 | AR | Aicardi-Goutieres syndrome (SAMHD1-related) |
| TREX1 | AR | Aicardi-Goutieres syndrome (TREX1-related) |
| TYRP1 | AR | albinism, oculocutaneous, type III |
| HGD | AR | alkaptonuria |
| SERPINA1 | AR | alpha-1 antitrypsin deficiency |
| MAN2B1 | AR | alpha-mannosidosis |
| HBA1 | AR | alpha-thalassemia |
| HBA2 | AR | alpha-thalassemia |
| ATRX | XL | alpha-thalassemia mental retardation syndrome |
| COL4A3 | AR | Alport syndrome (COL4A3-related) |
| COL4A4 | AR | Alport syndrome (COL4A4-related) |
| COL4A5 | XL | Alport syndrome (COL4A5-related, X-linked) |
| ALMS1 | AR | Alstrom syndrome |
| SLC12A6 | AR | Andermann syndrome |
| POR | AR | Antley-Bixler syndrome (POR-related) |
| ARG1 | AR | argininemia |
| ASL | AR | argininosuccinic aciduria |
| CYP19A1 | AR | aromatase deficiency |
| SLC35A3 | AR | arthrogryposis, mental retardation, and seizures |
| ASNS | AR | asparagine synthetase deficiency |
| AGA | AR | aspartylglycosaminuria |
| TTPA | AR | ataxia with isolated vitamin E deficiency |
| ATM | AR | ataxia-telangiectasia |
| MRE11 | AR | ataxia-telangiectasia-like disorder I |
| SACS | AR | autosomal recessive spastic ataxia of Charlevoix-Saguenay |
| ARL6 | AR | Bardet-Biedl syndrome (ARL6-related) |
| BBS10 | AR | Bardet-Biedl syndrome (BBS10-related) |
| BBS12 | AR | Bardet-Biedl syndrome (BBS12-related) |
| BBS1 | AR | Bardet-Biedl syndrome (BBS1-related) |
| BBS2 | AR | Bardet-Biedl syndrome (BBS2-related) |
| BBS4 | AR | Bardet-Biedl syndrome (BBS4-related) |
| CIITA | AR | bare lymphocyte syndrome, type II |
| TAZ | XL | Barth syndrome (X-linked) |
| CLCNKB | AR | Bartter syndrome, type 3 |
| BSND | AR | Bartter syndrome, type 4A |
| GP1BA | AR | Bernard-Soulier syndrome, type A1 |
| GP9 | AR | Bernard-Soulier syndrome, type C |
| HBB | AR | beta-globin-related hemoglobinopathies |
| ACAT1 | AR | beta-ketothiolase deficiency |
| MANBA | AR | beta-mannosidosis |
| QDPR | AR | BH4-deficient hyperphenylalaninemia C |
| PCBD1 | AR | BH4-deficient hyperphenylalaninemia D |
| GPR56 | AR | bilateral frontoparietal polymicrogyria |
| BTD | AR | biotinidase deficiency |
| BLM | AR | Bloom syndrome |
| GDF5 | AR | brachydactyly and other GDF5-related skeletal disorders |
| BCHE | AR | butyrylcholinesterase deficiency |
| ASPA | AR | Canavan disease |
| CPS1 | AR | carbamoylphosphate synthetase I deficiency |
| SLC25A20 | AR | carnitine acylcarnitine translocase deficiency |
| CPT1A | AR | carnitine palmitoyltransferase IA deficiency |
| CPT2 | AR | carnitine palmitoyltransferase II deficiency |
| RAB23 | AR | Carpenter syndrome |
| RMRP | AR | cartilage-hair hypoplasia |
| CASQ2 | AR | catecholaminergic polymorphic ventricular tachycardia |
| CD59 | AR | CD59-mediated hemolytic anemia |
| IGSF1 | XL | central hypothyroidism and testicular enlargement (X-linked) |
| GATM | AR | cerebral creatine deficiency syndrome (GATM-related) |
| SLC6A8 | XL | cerebral creatine deficiency syndrome 1 (X-linked) |
| GAMT | AR | cerebral creatine deficiency syndrome 2 |
| SNAP29 | AR | cerebral dysgenesis, neuropathy, ichthyosis, and palmoplantar |
| | | keratoderma syndrome |
| CYP27A1 | AR | cerebrotendinous xanthomatosis |
| NDRG1 | AR | Charcot-Marie-Tooth disease, type 4D |
| PRPS1 | XL | Charcot-Marie-Tooth disease, type 5 I Arts syndrome/deafness, |
| | | X-linked 1 |
| GJB1 | XL | Charcot-Marie-Tooth disease, X-linked |
| LYST | AR | Chediak-Higashi syndrome |
| ARSE | XL | chondrodysplasia punctata (X-linked) |
| VPS13A | AR | choreoacanthocytosis |
| CHM | XL | choroideremia (X-linked) |
| CYBA | AR | chronic granulomatous disease (CYBA-related) |
| CYBB | XL | chronic granulomatous disease (CYBB-related, X-linked) |
| SLC25A13 | AR | citrin deficiency |
| ASS1 | AR | citrullinemia, type 1 |
| ERCC8 | AR | Cockayne syndrome, type A |
| ERCC6 | AR | Cockayne syndrome, type Band other ERCC6-related disorders |
| VPS13B | AR | Cohen syndrome |
| LMAN1 | AR | combined factor V and VIII deficiency |
| ACSF3 | AR | combined malonic and methylmalonic aciduria |
| GFM1 | AR | combined oxidative phosphorylation deficiency 1 |
| TSFM | AR | combined oxidative phosphorylation deficiency 3 |
| POU1F1 | AR | combined pituitary hormone deficiency 1 |
| PROP1 | AR | combined pituitary hormone deficiency 2 |
| LHX3 | AR | combined pituitary hormone deficiency 3 |
| PSAP | AR | combined SAP deficiency |
| GUCY2D | AR | cone-rod dystrophy 6/Leber congenital amaurosis 1 |
| CYP11B1 | AR | congenital adrenal hyperplasia due to 11-beta-hydroxylase |
| | | deficiency |
| CYP17A1 | AR | congenital adrenal hyperplasia due to 17-alpha-hydroxylase |
| | | deficiency |
| CYP21A2 | AR | congenital adrenal hyperplasia due to 21-hydroxylase deficiency |
| NR0B1 | XL | congenital adrenal hypoplasia (NR0B1 -related, X-linked) |
| CYP11A1 | AR | congenital adrenal insufficiency (CYP11A1-related) |
| MPL | AR | congenital amegakaryocytic thrombocytopenia |
| AKR1D1 | AR | congenital bile acid synthesis defect (AKR1D1-related) |
| HSD3B7 | AR | congenital bile acid synthesis defect (HSD3B7-related) |
| NGLY1 | AR | congenital disorder of deglycosylation |
| PMM2 | AR | congenital disorder of glycosylation, type Ia |
| MPI | AR | congenital disorder of glycosylation, type Ib |
| ALG6 | AR | congenital disorder of glycosylation, type Ie |
| DOLK | AR | congenital disorder of glycosylation, type Im |
| SEC23B | AR | congenital dyserythropoietic anemia type 2 |
| CDAN1 | AR | congenital dyserythropoietic anemia, type 1a |
| ABCA12 | AR | congenital ichthyosis 4A and 4B |
| NTRK1 | AR | congenital insensitivity to pain with anhidrosis |
| LAMA2 | AR | congenital muscular dystrophy (LAMA2-related) |
| CHAT | AR | congenital myasthenic syndrome (CHAT-related) |
| CHRNE | AR | congenital myasthenic syndrome (CHRNE-related) |
| DOK? | AR | congenital myasthenic syndrome (DOK7-related) |
| RAPSN | AR | congenital myasthenic syndrome (RAPSN-related) |
| HAX1 | AR | congenital neutropenia (HAX1-related) |
| VPS45 | AR | congenital neutropenia (VPS45-related) |
| TSHR | AR | Congenital nongoitrous hypothyroidism 1Inonautoim |
| | | munehyperthyroidis |
| TSHB | AR | congenital nongoitrous hypothryoidism 4 |
| SLC26A3 | AR | congenital secretory chloride diarrhea 1 |
| SLC4A11 | AR | corneal dystrophy and perceptive deafness |
| CYP11 B2 | AR | corticosterone methyloxidase deficiency |
| UGT1A1 | AR | Crigler-Najjar syndrome, types 1 & 2/Gilbert syndrome |
| CFTR | AR | cystic fibrosis |
| CTNS | AR | Cystinosis |
| SLC3A1 | AR | cystinuria (SLC3A1-related) |
| COX15 | AR | cytochrome c oxidase deficiency/Leigh syndrome (COX15- |
| | | related) |
| HSD17B4 | AR | D-bifunctional protein deficiency |
| MY015A | AR | deafness, autosomal recessive 3 |
| PJVK | AR | deafness, autosomal recessive 59 |
| TMC1 | AR | deafness, autosomal recessive 7 |
| SYNE4 | AR | deafness, autosomal recessive 76 |
| LOXHD1 | AR | deafness, autosomal recessive 77 |
| TMPRSS3 | AR | deafness, autosomal recessive 8/10 |
| OTOF | AR | deafness, autosomal recessive 9 |
| CANT1 | AR | Desbuquois dysplasia 1 |
| DHCR24 | AR | Desmosterolosis |
| BMPER | AR | Diaphanospondylodysostosis |
| OPYD | AR | dihydropyrimidine dehydrogenase deficiency/5-fluorouracil |
| | | toxicity |
| SLC4A1 | AR | distal renal tubular acidosis/spherocytosis, type 4 |
| DMD | XL | Duchenne muscular dystrophy/Becker muscular dystrophy (X- |
| | | linked) |
| RTEL1 | AR | dyskeratosis congenita (RTEL1-related) |
| DKC1 | XL | dyskeratosis congenita (X-linked) |
| COL7A1 | AR | dystrophic epidermolysis bullosa |
| PLOD1 | AR | Ehlers-Danlos syndrome, type VI |
| ADAMTS2 | AR | Ehlers-Danlos syndrome, type VIIC |
| EVC2 | AR | Ellis-van Creveld syndrome (EVC2-related) |
| EVC | AR | Ellis-van Creveld syndrome (EVC-related) |
| EMO | XL | Emery-Dreifuss myopathy 1 (X-linked) |
| NR2E3 | AR | enhanced S-cone syndrome |
| ETHE1 | AR | ethylmalonic encephalopathy |
| GLA | XL | Fabry disease (X-linked) |
| F9 | XL | factor IX deficiency (X-linked) |
| F7 | AR | factor VII deficiency |
| F11 | AR | factor XI deficiency |
| LDLRAP1 | AR | familial autosomal recessive hypercholesterolemia |
| IKBKAP | AR | familial dysautonomia |
| LDLR | AR | familial hypercholesterolemia |
| HADH | AR | familial hyperinsulinemic hypoglycemia 4/3-hydroxyacyl-CoA |
| | | dehydrogenase deficiency |
| ABCC8 | AR | familial hyperinsulinism (ABCC8-related) |
| KCNJ11 | AR | familial hyperinsulinism (KCNJ11-related) |
| GALNT3 | AR | familial hyperphosphatemic tumoral calcinosis |
| MEFV | AR | familial Mediterranean fever |
| FANCA | AR | Fanconi anemia, group A |
| FANCC | AR | Fanconi anemia, group C |
| FANCG | AR | Fanconi anemia, group G |
| SLC2A2 | AR | Fanconi-Bickel syndrome |
| FMR1 | XL | fragile X syndrome |
| FBP1 | AR | fructose-1,6-bisphosphatase deficiency |
| FUCA1 | AR | Fucosidosis |
| FH | AR | fumarase deficiency |
| RDH5 | AR | fundus albipunctatus |
| GALK1 | AR | galactokinase deficiency |
| GALE | AR | galactose epimerase deficiency |
| GALT | AR | galactosemia |
| CTSA | AR | Galactosialidosis |
| GBA | AR | Gaucher disease |
| TRHR | AR | generalized thyrotropin-releasing hormone resistance |
| GORAB | AR | geroderma osteodysplasticum |
| SLC12A3 | AR | Gitelman syndrome |
| ITGA2B | AR | Glanzmann thrombasthenia (ITGA2B-related) |
| ITGB3 | AR | Glanzmann thrombasthenia (ITGB3-related) |
| GCDH | AR | glutaric acidemia, type I |
| ETFA | AR | glutaric acidemia, type IIa |
| ETFB | AR | glutaric acidemia, type IIb |
| ETFDH | AR | glutaric acidemia, type IIc |
| GSS | AR | glutathione synthetase deficiency |
| AMT | AR | glycine encephalopathy (AMT-related) |
| GLDC | AR | glycine encephalopathy (GLDC-related) |
| GYS2 | AR | glycogen storage disease, type 0 |
| G6PC | AR | glycogen storage disease, type Ia |
| SLC37A4 | AR | glycogen storage disease, type Ib |
| GAA | AR | glycogen storage disease, type II |
| AGL | AR | glycogen storage disease, type III |
| GBE1 | AR | glycogen storage disease, type IV/adult polyglucosan body |
| | | disease |
| PHKB | AR | glycogen storage disease, type IXb |
| PYGM | AR | glycogen storage disease, type V |
| PYGL | AR | glycogen storage disease, type VI |
| PFKM | AR | glycogen storage disease, type VII |
| BCS1L | AR | GRACILE syndrome and other BCS1L-related disorders |
| NBEAL2 | AR | gray platelet syndrome |
| GHRHR | AR | growth hormone deficiency, type IB |
| HFE | AR | hemochromatosis, type 1 |
| HFE2 | AR | hemochromatosis, type 2A |
| TFR2 | AR | hemochromatosis, type 3 |
| G6PD | XL | hemolytic anemia (G6PD-related, X-linked) |
| ALDOB | AR | hereditary fructose intolerance |
| TECPR2 | AR | hereditary spastic paraparesis 49 |
| HPS1 | AR | Hermansky-Pudlak syndrome, type 1 |
| HPS3 | AR | Hermansky-Pudlak syndrome, type 3 |
| HPS4 | AR | Hermansky-Pudlak syndrome, type 4 |
| HPS6 | AR | Hermansky-Pudlak syndrome, type 6 |
| HMGCL | AR | HMG-CoA lyase deficiency |
| HMGCS2 | AR | HMG-CoA synthase 2 deficiency |
| HLCS | AR | holocarboxylase synthetase deficiency |
| CBS | AR | homocystinuria (CBS-related) |
| MTHFR | AR | homocystinuria due to MTHFR deficiency |
| MTRR | AR | homocystinuria, cbIE type |
| MTR | AR | homocystinuria-megaloblastic anemia, cobalamin G type |
| L1CAM | XL | hydrocephalus (X-linked) |
| HYLS1 | AR | hydrolethalus syndrome |
| CD40LG | XL | hyper-IgM syndrome (X-linked) |
| SLC25A15 | AR | hyperomithinemia-hyperammonemia-homocitru11inuria |
| | | syndrome |
| SARS2 | AR | hyperuricemia, pulmonary hypertension, renal failure, and |
| | | alkalosis |
| EDA | XL | hypohidrotic ectodermal dysplasia 1 (X-linked) |
| TRPM6 | AR | hypomagnesemia 1 |
| AIMP1 | AR | hypomyelinating leukodystrophy 3 |
| VPS11 | AR | hypomyelinating leukodystrophy 12 |
| TBCE | AR | hypoparathyroidism-retardation-dysmorphic syndrome |
| ALPL | AR | Hypophosphatasia |
| SLC34A3 | AR | hypophosphatemic rickets with hypercalciuria |
| LPAR6 | AR | hypotrichosis 8/autosomal recessive woolly hair 1 |
| CD3E | AR | immunodeficiency 18 |
| CD3D | AR | immunodeficiency 19 |
| GNE | AR | inclusion body myopathy 2 |
| MED17 | AR | infantile cerebral and cerebellar atrophy |
| PLA2G6 | AR | infantile neuroaxonal dystrophy 1 and other PLA2G6-related |
| | | disorders |
| ATP8B1 | AR | intrahepatic cholestasis |
| IVD | AR | isovaleric acidemia |
| TMEM216 | AR | Joubert syndrome 2 |
| NPHP1 | AR | Joubert syndrome 4 Senior-Loken syndrome 1/Juvenile |
| | | nepronophthisis 1 |
| RPGRIP1L | AR | Joubert syndrome 7/Meckel syndrome 5/COACH syndrome |
| COL17A1 | AR | junctional epidermolysis bullosa (COL17A1-related) |
| ITGA6 | AR | junctional epidermolysis bullosa (ITGA6-related) |
| ITGB4 | AR | junctional epidermolysis bullosa (ITGB4-related) |
| LAMA3 | AR | junctional epidermolysis bullosa (LAMA3-related) |
| LAMB3 | AR | junctional epidermolysis bullosa (LAMB3-related) |
| LAMC2 | AR | junctional epidermolysis bullosa (LAMC2-related) |
| ROGOi | AR | Kohlschutter-Tonz syndrome |
| GALC | AR | Krabbe disease |
| TGM1 | AR | lamellar ichthyosis, type 1 |
| GHR | AR | Laron dwarfism |
| CEP290 | AR | Leber congenital amaurosis 10 and other CEP290-related |
| | | ciliopathies |
| RDH12 | AR | Leber congenital amaurosis 13 |
| TULP1 | AR | Leber congenital amaurosis 15/retinitis pigmentosa 14 |
| RPE65 | AR | Leber congenital amaurosis 2/retinitis pigmentosa 20 |
| AIPL1 | AR | Leber congenital amaurosis 4 |
| LCA5 | AR | Leber congenital amaurosis 5 |
| CRB1 | AR | Leber congenital amaurosis 8/retinitis pigmentosa 12/ |
| | | pigmented paravenous chorioretinal atrophy |
| NDUFS7 | AR | Leigh syndrome (NDUFS7-related) |
| SURF1 | AR | Leigh syndrome (SURF1-related) |
| LRPPRC | AR | Leigh syndrome, French-Canadian type |
| GLE1 | AR | lethal congenital contracture syndrome 1/lethal arthrogryposis |
| | | with anterior horn cell disease |
| ERBB3 | AR | lethal congenital contracture syndrome 2 |
| PIP5K1C | AR | lethal congenital contracture syndrome 3 |
| EIF2B5 | AR | leukoencephalopathy with vanishing white matter |
| CAPN3 | AR | limb-girdle muscular dystrophy, type 2A |
| DYSF | AR | limb-girdle muscular dystrophy, type 2B |
| SGCG | AR | limb-girdle muscular dystrophy, type 2C |
| SGCA | AR | limb-girdle muscular dystrophy, type 2D |
| SGCB | AR | limb-girdle muscular dystrophy, type 2E |
| SGCD | AR | limb-girdle muscular dystrophy, type 2F |
| TRIM32 | AR | limb-girdle muscular dystrophy, type 2H |
| FKRP | AR | limb-girdle muscular dystrophy, type 21 |
| ANOS | AR | limb-girdle muscular dystrophy, type 2L |
| OLD | AR | lipoamide dehydrogenase deficiency |
| STAR | AR | lipoid adrenal hyperplasia |
| LPL | AR | lipoprotein lipase deficiency |
| HADHA | AR | long-chain 3-hydroxyacyl-CoA dehydrogenase deficiency |
| OCRL | XL | Lowe syndrome (X-linked) |
| SLC7A7 | AR | lysinuric protein Intolerance |
| LHCGR | AR | male precocious puberty and other LHCGR-related disorders |
| HSD17B3 | AR | male pseudohermaphroditism with gynecomastia |
| RYR1 | AR | malignant hyperthermia and other RYR1-related myopathies |
| MLYCD | AR | malonyl-CoA decarboxylase deficiency |
| BCKDHA | AR | maple syrup urine disease, type 1a |
| BCKDHB | AR | maple syrup urine disease, type 1b |
| DBT | AR | maple syrup urine disease, type 2 |
| MKS1 | AR | Meckel syndrome 1/Bardet-Biedl syndrome 13 |
| ACADM | AR | medium chain acyl-CoA dehydrogenase deficiency |
| AP1S1 | AR | MEDNIK syndrome |
| MLC1 | AR | megalencephalic leukoencephalopathy with subcortical cysts |
| AMN | AR | megaloblastic anemia 1 |
| ATP7A | XL | Menkes disease |
| CC2D1A | AR | mental retardation, autosomal recessive 3 |
| ARSA | AR | metachromatic leukodystrophy |
| MAT1A | AR | methionine adenosyltransferase I/III deficiency |
| MMAA | AR | methylmalonic acidemia (MMAA-related) |
| MMAB | AR | methylmalonic acidemia (MMAB-related) |
| MUT | AR | methylmalonic acidemia (MUT-related) |
| MMACHC | AR | methylmalonic aciduria and homocystinuria, cobalamin C type |
| MMADHC | AR | methylmalonic aciduria and homocystinuria, cobalamin D type |
| LMBRD1 | AR | methylmalonic aciduria and homocystinuria, cobalamin F type |
| MCEE | AR | methylmalonyl-CoA epimerase deficiency |
| VSX2 | AR | microphthalmia/anophthalmia |
| ACAD9 | AR | mitochondrial complex I deficiency (ACAD9-related) |
| NDUFA11 | AR | mitochondrial complex I deficiency (NDUFA11-related) |
| NDUFAF5 | AR | mitochondrial complex I deficiency (NDUFAF5-related) |
| NDUFS6 | AR | mitochondrial complex I deficiency (NDUFS6-related) |
| NDUFV1 | AR | mitochondrial complex I deficiency (NDUFV1-related) |
| FOXRED1 | AR | mitochondrial complex I deficiency/Leigh syndrome |
| | | (FOXRED1-related) |
| NDUFAF2 | AR | mitochondrial complex I deficiency/Leigh syndrome |
| | | (NDUFAF2-related) |
| NDUFS4 | AR | mitochondrial complex I deficiency/Leigh syndrome (NDUFS4- |
| | | related) |
| COX20 | AR | mitochondrial complex IV deficiency (COX20-related) |
| COX6B1 | AR | mitochondrial complex IV deficiency (COX6B1-related) |
| APOPT1 | AR | mitochondrial complex IV deficiency (APOPT1-related) |
| PET100 | AR | mitochondrial complex IV deficiency (PET1DO-related) |
| SCO1 | AR | mitochondrial complex IV deficiency (SCO1-related) |
| COX10 | AR | mitochondrial complex IV deficiency/Leigh Syndrome (COX10- |
| | | related) |
| TK2 | AR | mitochondrial DNA depletion syndrome 2 |
| DGUOK | AR | mitochondrial DNA depletion syndrome 3 |
| POLG | AR | mitochondrial DNA depletion syndrome 4A and 4B and other |
| | | POLG-related disorders |
| SUCLA2 | AR | mitochondrial DNA depletion syndrome 5 |
| MPV17 | AR | mitochondrial DNA depletion syndrome 6 I Navajo |
| | | neurohepatopathy |
| PUS1 | AR | mitochondrial myopathy and sideroblastic anemia 1 |
| HADHB | AR | mitochondrial trifunctional protein deficiency (HADHB-related) |
| MOCS1 | AR | molybdenum cofactor deficiency A |
| GNPTAB | AR | mucolipidosis II/IIIA |
| GNPTG | AR | mucolipidosis Ill gamma |
| MCOLN1 | AR | mucolipidosis IV |
| IDUA | AR | mucopolysaccharidosis type I |
| IDS | XL | mucopolysaccharidosis type II |
| SGSH | AR | mucopolysaccharidosis type IIIA |
| NAGLU | AR | mucopolysaccharidosis type IIIB |
| HGSNAT | AR | mucopolysaccharidosis type IIIC |
| GNS | AR | mucopolysaccharidosis type HID |
| GALNS | AR | mucopolysaccharidosis type IVa |
| GLB1 | AR | mucopolysaccharidosis type IVb/GM1 gangliosidosis |
| ARSB | AR | mucopolysaccharidosis type VI |
| GUSB | AR | mucopolysaccharidosis VII |
| HYAL1 | AR | mucopolysaccharidosis type IX |
| TRIM37 | AR | mulibrey nanism |
| PIGN | AR | multiple congenital anomalies-hypotonia-seizures syndrome 1 |
| CHRNG | AR | multiple pterygium syndrome |
| SUMF1 | AR | multiple sulfatase deficiency |
| POMGNT1 | AR | muscle-eye-brain disease and other POMGNT1 -related |
| | | congenital muscular dystrophy-dystroglycanopathies |
| TYMP | AR | myoneurogastrointestinal encephalopathy |
| MTM1 | XL | myotubular myopathy 1 (X-linked) |
| NAGS | AR | N-acetylglutamate synthase deficiency |
| NEB | AR | nemaline myopathy 2 |
| AVPR2 | XL | nephrogenic diabetes insipidus (AVPR2-related)/nephrogenic |
| | | syndrome (X-linked) |
| AQP2 | AR | nephrogenic diabetes insipidus, type II |
| INVS | AR | nephronophthisis 2 |
| NPHS1 | AR | nephrotic syndrome (NPHS1-related) I congenital Finnish |
| | | nephrosis |
| NPHS2 | AR | nephrotic syndrome (NPHS2-related)/steroid-resistant nephrotic |
| | | syndrome |
| FOLR1 | AR | neurodegeneration due to cerebral folate transport deficiency |
| CLN3 | AR | neuronal ceroid-lipofuscinosis (CLN3-related) |
| CLN5 | AR | neuronal ceroid-lipofuscinosis (CLN5-related) |
| CLN6 | AR | neuronal ceroid-lipofuscinosis (CLN6-related) |
| CLN8 | AR | neuronal ceroid-lipofuscinosis (CLN8-related) |
| MFSD8 | AR | neuronal ceroid-lipofuscinosis (MFSD8-related) |
| PPT1 | AR | neuronal ceroid-lipofuscinosis (PPT1-related) |
| TPP1 | AR | neuronal ceroid-lipofuscinosis (TPP1-related) |
| SMPD1 | AR | Niemann-Pick disease (SMPD1-related) |
| NPC1 | AR | Niemann-Pick disease, type C (NPC1-related) |
| NPC2 | AR | Niemann-Pick disease, type C (NPC2-related) |
| NBN | AR | Nijmegen breakage syndrome |
| GJB2 | AR | non-syndromic hearing loss (GJB2-related) |
| TYR | AR | oculocutaneous albinism, type IA/IB |
| SLC45A2 | AR | oculocutaneous albinism, type IV |
| WNT10A | AR | odonto-onycho-dermal dysplasia/Schopf-Schulz-Passarge |
| | | syndrome |
| RAG2 | AR | Omenn syndrome (RAG2-related) |
| DCLRE1C | AR | Omenn syndrome I severe combined immunodeficiency, |
| | | Athabaskan-type |
| RAG1 | AR | Omenn syndrome and other RAG1-related disorders |
| OAT | AR | ornithine aminotransferase deficiency |
| OTC | XL | ornithine transcarbamylase deficiency (X-linked) |
| FKBP10 | AR | osteogenesis imperfecta, type XI |
| TCIRG1 | AR | osteopetrosis 1 |
| SNX10 | AR | osteopetrosis 8 |
| COL11A2 | AR | otospondylomegaepiphyseal dysplasia/deafness/ |
| | | fibrochondrogenesis 2 |
| CTSC | AR | Papillon-Lefevre syndrome |
| SLC26A4 | AR | Pendred syndrome |
| PEX12 | AR | peroxisome biogenesis disorder 3 A and 3B |
| PEX26 | AR | peroxisome biogenesis disorder 7A and 7B |
| AMH | AR | persistent Mullerian duct syndrome, type I |
| AMHR2 | AR | persistent Mullerian duct syndrome, type II |
| PAH | AR | phenylalanine hydroxylase deficiency |
| PLAA | AR | PLAA-related neurodevelopmental disorders |
| PKHD1 | AR | polycystic kidney disease, autosomal recessive |
| AIRE | AR | polyglandular autoimmune syndrome, type 1 |
| VRK1 | AR | pontocerebellar hypoplasia, type 1A |
| EXOSC3 | AR | pontocerebellar hypoplasia, type 1B |
| TSEN54 | AR | pontocerebellar hypoplasia, type 2A and type 4 |
| VPS53 | AR | pontocerebellar hypoplasia, type 2E |
| RARS2 | AR | pontocerebellar hypoplasia, type 6 |
| SLC22A5 | AR | primary carnitine deficiency |
| CCDC103 | AR | primary ciliary dyskinesia (CCDC103-related) |
| CCDC151 | AR | primary ciliary dyskinesia (CCDC151-related) |
| CCDC39 | AR | primary ciliary dyskinesia (CCDC39-related) |
| DNAH5 | AR | primary ciliary dyskinesia (DNAH5-related) |
| DNAl1 | AR | primary ciliary dyskinesia (DNAl1-related) |
| DNAl2 | AR | primary ciliary dyskinesia (DNA12-related) |
| RSPH9 | AR | primary ciliary dyskinesia (RSPH9-related) |
| COQ4 | AR | primary coenzyme 010 deficiency 7 |
| CYP1B1 | AR | primary congenital glaucoma |
| AGXT | AR | primary hyperoxaluria, type 1 |
| GRHPR | AR | primary hyperoxaluria, type 2 |
| HOGA1 | AR | primary hyperoxaluria, type 3 |
| SEPSECS | AR | progressive cerebello-cerebral atrophy |
| ABCB11 | AR | progressive familial intrahepatic cholestasis, type 2 |
| PRICKLE1 | AR | progressive myoclonic epilepsy, type 1B |
| WISP3 | AR | progressive pseudorheumatoid dysplasia |
| PEPD | AR | prolidase deficiency |
| PCCA | AR | propionic acidemia (PCCA-related) |
| PCCB | AR | propionic acidemia (PCCB-related) |
| SRD5A2 | AR | pseudovaginal perineoscrotal hypospadias |
| ABCA3 | AR | pulmonary surfactant dysfunction |
| CTSK | AR | Pycnodysostosis |
| PNPO | AR | pyridoxamine 5′-phosphate oxidase deficiency |
| ALDH7A1 | AR | pyridoxine-dependent epilepsy |
| PC | AR | pyruvate carboxylase deficiency |
| PDHA1 | XL | pyruvate dehydrogenase E1-alpha deficiency (X-linked) |
| PDHB | AR | pyruvate dehydrogenase E1-beta deficiency |
| ATP6V1B1 | AR | renal tubular acidosis and deafness |
| EYS | AR | retinitis pigmentosa 25 |
| CERKL | AR | retinitis pigmentosa 26 |
| FAM161A | AR | retinitis pigmentosa 28 |
| PRCD | AR | retinitis pigmentosa 36 |
| DHDDS | AR | retinitis pigmentosa 59 |
| C8ORF37 | AR | retinitis pigmentosa 64/Bardet-Biedl syndrome 21/cone-rod |
| | | dystrophy 16 |
| RLBP1 | AR | retinitis punctata albescens and other RLBP1-related ocular |
| | | disorders |
| RHAG | AR | Rh deficiency syndrome |
| PEX7 | AR | rhizomelic chondrodysplasia punctata, type 1 |
| AGPS | AR | rhizomelic chondrodysplasia punctata, type 3 |
| ESCO2 | AR | Roberts syndrome |
| SLC17A5 | AR | Salla disease |
| ST3GAL5 | AR | salt and pepper developmental regression syndrome |
| HEXB | AR | Sandhoff disease |
| SMARCAL1 | AR | Schimke immunoosseous dysplasia |
| CEP152 | AR | Seckel syndrome 5/microcephaly 9 |
| TH | AR | Segawa syndrome |
| SPR | AR | sepiapterin reductase deficiency |
| IL7R | AR | severe combined immunodeficiency (IL7R-related) |
| JAK3 | AR | severe combined immunodeficiency (JAK3-related) |
| PTPRC | AR | severe combined immunodeficiency (PTPRC-related) |
| G6PC3 | AR | severe congenital neutropenia 4 |
| CASR | AR | severe neonatal hyperparathyroidism |
| POC1A | AR | short stature, onychodysplasia, facial dysmorphism, and |
| | | hypotrichosis |
| ACADS | AR | short-chain acyl-CoA dehydrogenase deficiency |
| SBDS | AR | Shwachman-Diamond syndrome |
| NEU1 | AR | sialidosis, type I and type II |
| ALDH3A2 | AR | Sjogren-Larsson syndrome |
| DHCR7 | AR | Smith-Lemli-Opitz syndrome |
| ZFYVE26 | AR | spastic paraplegia 15 |
| SLC1A4 | AR | spastic tetraplegia, thin corpus callosum, and progressive |
| | | microcephaly |
| EPB42 | AR | spherocytosis, type 5 |
| SMN1 | AR | spinal muscular atrophy |
| IGHMBP2 | AR | spinal muscular atrophy with respiratory distress 1/Charcot- |
| | | Marie-Tooth disease, type 2 |
| COA7 | AR | spinocerebellar ataxia with axonal neuropathy 3 |
| DLL3 | AR | spondylocostal dysostosis 1 |
| DDR2 | AR | spondylometaepiphyseal dysplasia (DDR2-related) |
| MESP2 | AR | spondylothoracic dysostosis |
| ABCA4 | AR | Stargardt disease and other ABCA4-related ocular disorders |
| COL27A1 | AR | Steel syndrome |
| LIFR | AR | Stuve-Wiedemann syndrome |
| SLC26A2 | AR | sulfate transporter-related osteochondrodysplasia |
| HEXA | AR | Tay-Sachs disease |
| SLC19A2 | AR | thiamine-responsive megaloblastic anemia syndrome |
| F2 | AR | thrombophilia/factor II deficiency |
| F5 | AR | thrombophilia/factor V deficiency |
| SLC5A5 | AR | thyroid dyshormonogenesis 1 |
| TPO | AR | thyroid dyshormonogenesis 2A |
| TG | AR | thyroid dyshormonogenesis 3 |
| IYD | AR | thyroid dyshormonogenesis 4 |
| DUOXA2 | AR | thyroid dyshormonogenesis 5 |
| DUOX2 | AR | thyroid dyshormonogenesis 6 |
| TTC37 | AR | trichohepatoenteric syndrome 1 |
| FAH | AR | tyrosinem ia, type I |
| TAT | AR | tyrosinem ia, type 11 |
| HPD | AR | tyrosinem ia, type 111/hawkinsinuria |
| MYO7A | AR | Usher syndrome, type IB |
| USH1C | AR | Usher syndrome, type IC |
| CDH23 | AR | Usher syndrome, type ID |
| PCDH15 | AR | Usher syndrome, type IF |
| USH2A | AR | Usher syndrome, type IIA |
| CLRN1 | AR | Usher syndrome, type Ill |
| ACADVL | AR | very long chain acyl-CoA dehydrogenase deficiency |
| CYP27B1 | AR | vitamin D-dependent rickets, type I |
| VDR | AR | vitamin D-resistant rickets, type IIA |
| VWF | AR | van Willebrand disease |
| FKTN | AR | Walker-Warburg syndrome and other FKTN-related dystrophies |
| WRN | AR | Werner syndrome |
| ATP7B | AR | Wilson disease |
| WAS | XL | Wiskott-Aldrich syndrome (WAS-related, X-linked) |
| EIF2AK3 | AR | Wolcott-Rallison syndrome |
| LIPA | AR | Wolman disease/cholesteryl ester storage disease |
| DCAF17 | AR | Woodhouse-Sakati syndrome |
| POLH | AR | xeroderma pigmentosum (POLH-related) |
| XPA | AR | xeroderma pigmentosum, group A |
| XPC | AR | xeroderma pigmentosum, group C |
| ERCC5 | AR | xeroderma pigmentosum, group G |
| RS1 | XL | X-linked juvenile retinoschisis |
| IL2RG | XL | X-linked severe combined immunodeficiency |
| PEX10 | AR | Zellweger syndrome spectrum (PEX10-related) |
| PEX1 | AR | Zellweger syndrome spectrum (PEX1-related) |
| PEX2 | AR | Zellweger syndrome spectrum (PEX2-related) |
| PEX6 | AR | Zellweger syndrome spectrum (PEX6-related) |

### EXAMPLE RESIDUAL RISK DETERMINATION PROCESS

FIG. 4 is a flowchart depicting an example residual risk determination process 400, in accordance with some embodiments. The process 400 may be performed by a computing device, such as the computing server 130. The process 400 may correspond to step 220 through step 245 discussed in FIG. 2. The process 400 may be used to determine the residual risk of an individual being a carrier of a genetic disease or to determine the risk of a prospective offspring having the genetic disease. The residual risk value for each genetic disease may be different, especially for various ethnicity. The residual risk may correspond to the probability or risk of an offspring inheriting a given disease or condition based upon a given set of genetic data, after correcting for or reducing the risk based on factors including such as molecular ancestry. For the same individual, the process 400 may be repeated for different genetic diseases.

A computing device retrieves 410 an individual profile for an individual and a sequence dataset associated with the individual profile. The sequence dataset may be the result of sequencing the second set of nucleic acid samples as discussed in step 220 in FIG. 2. For example, the sequencing dataset may be the result of a low-pass whole genome sequencing that covers at least a substantial portion of the genome but has a low coverage depth. In some embodiments, the nucleic acid samples may be randomly cleaved. The genomic locations may be randomly sampled and sequenced so that the sequence dataset for one individual has different genomic regions that another individual. The sequencing may be carried out by the sequencing system 120, as discussed in FIG. 1. The sequence dataset is associated with the individual profile, but the sequence dataset does not always need to be sequenced from a biological sample of the individual. For example, in one case, the sequence dataset is sequenced from the biological sample of the individual. In another case, the sequence dataset is sequenced from the biological sample of a relative of the individual. In yet another case, the individual is a prospective offspring and the sequence dataset belongs to one of the prospective parents.

The computing device may determine 420 an ancestral composition of the sequence dataset. The determination of ancestral composition may include comparing the sequence dataset to a library of ancestry-specific reference sets, which may be retrieved from one or more biomarker data servers 150. For a particular reference set, the sequence dataset, which may include randomly selected genomic locations, is aligned against the reference set. Once aligned, base calling is performed to identify any SNPs present in the sequence dataset. After base calling, the identified SNPs are used to perform global ancestry analysis that assigns the global ancestry of the individual. The comparison may be repeated for other reference sets. Each reference set may have a different degree of alignment with the sequence dataset. The ancestral composition may be determined based on the degree of similarities of SNPs between the sequence dataset and the various reference sets.

The ancestral composition may be detremiend using sequencing data based on various sequencing techniques. In one embodiments, a small number of SNPs (e.g., in the magnitude of hundreds of SNPs or as few as about 82 SNPs) may be used for ancestry definition. Ligation-dependent probe amplification (MLPA), SNPlex from APPLIED BIOSYSTEMS (ABI), AGENA MALDI-TOF genotyping, LUMINEX, or suitable Sanger sequencing techniques may be used to generated a small number of SNPs. Other arrays can be used to generate a larger number of SNPs (e.g., hundreds of thousands or millions), such as AFFYMETRIX array, AGILENT SNP arranys, ILLUMINA INFINIUM. The ancestral composition may also be generated based on NGS sequencing data. Various techniques may be used to generate libraries for NGS such as COVARIS physical shearing with any adapters, Enzymatic shearing methods from ILLUMINA (NEXTERA), AGILENT, KAPA/ROCHE. Targeted sequencing may be used for global ancestry determination. For example, global ancestry may be determined from datat of targeted sequencing using on and off target data. In some embodiements, low-pass sequencing discussed in this disclosure may be used to determine ancestral compositions. In other embodiments, high-resolution sequencing may be used to determine ancestral compositions. In yet other embodiments, high-resolution whole genome sequencing may be used to determine ancestral compositions.

The ancestry pipeline of computing server 130 infers the global ancestry for each individual sample. The ancestry pipeline may include a wrapper program to integrate the ancestry composition algorithm with other widely used open source software and an in-house highly curated reference set of 3.3M+SNPs in a worldwide reference panel of 7,345 individuals grouped together into 49 populations. In some embodiments, the computing server 130 may collapse the reference panel into 26 broader ethnic groups to represent the ancestry composition at a higher level. Concurrently, these 49 populations are also binned into 8 groups (7 major ancestries plus an unassigned group) to match the populations present in the gnomAD public database which are used as reference for the residual risk calculation.

By way of example, the raw input genetic data is generated from a low-pass sequencing. The DNA is extracted from the collected samples and submitted for low-pass sequencing on the Illumina Platform which is a high-throughput whole-genome solution where the genome is shotgun sequenced (a method that involves breaking the genome into a collection of small DNA fragments) at a low coverage across the genome (most frequently between 0.4× and 1×).

The resulting FASTQ data file (a text-based format for storing biological sequence, called reads, and its quality scores) is further processed through a series of genomic algorithms and software to perform: 1) alignment against the human reference genome (hg19) and 2) variant calling. The alignment and variant calling analysis are both performed using open source software packages: BWA (Burrows-Wheeler Aligner) and SAMtools (which is a set of utilities that manipulate alignments). The output from these two analysis steps are represented in two different file formats: BAM (binary tab-delimited format that contains the information on sequence alignments) & Pileup file format (which describes the base-pair information at each chromosomal position). A minimal threshold number of 8 million reads from a sample may be set for a quality control analysis and of which, at least 75% need to be mapped to the reference genome. After the completion of these steps, the final data file in Pileup format is submitted to an ancestry composition determination algorithm. For BWA and SAMtools, Li, H., and Durbin, R. (2009), Fast and accurate short read alignment with Burrows-Wheeler transform, Bioinformatics 25,1754-1760 and Li H, Handsaker B, Wysoker A, et al., the Sequence Alignment/Map format and SAMtools, Bioinformatics. 2009;25(16):2078-2079. doi:10.1093/bioinformatics/btp352, are incorporated by reference for all purposes.

The ancestry composition determination algorithm uses a model-based clustering method to infer population structure and assign individuals to populations from multilocus genotype data. At a broad level, population structure is the existence of differing levels of genetic relatedness among some subgroups within a sample. This may arise for a variety of reasons, but a common cause is that samples have been drawn from geographically isolated groups or different locations across a geographic continuum. The model-based clustering algorithm identifies subgroups that have distinctive allele frequencies (a measure of the relative frequency of a genetic variant at a particular position in a group). This approach places individuals into K clusters, where K can be chosen in advance. The reference panel will be then used to identify these K clusters which in our case is defined as 49. As a result, individual samples can have membership in only one or more clusters (for admixed samples), with membership coefficients summing to 1 across clusters. In the worldwide sample, individuals from the same population nearly always shared similar membership coefficients in inferred clusters.

The ancestry composition determination algorithm assigns the ancestry proportions (membership coefficients) averaged across the genome of an individual (also known as global ancestry) from large autosomal SNP genotype datasets. The reference panel has ˜3M variants and each analysis uses a random subset of 150K SNPs and a total of 10 bootstraps are performed. A single bootstrap generates a ‘.Q’ file which contains the ancestry fractions inferred for the sample. An average of the ancestry proportion values from each of these 10 bootstraps is used as the final result. Afterwards, the ancestry composition determination algorithm summarizes all of the generated data into 2 different ancestry reports: 1) ancestry_high (with information for the 8 main groups) and 2) ancestry_low (with detailed ancestry information for the 26 ethnicity groups). And the report file that contains ancestry_high values is further integrated with the analysis that performs personalized residual risk (PPR) calculation. For further details of the ancestry composition determination algorithm, Pritchard J K, Stephens M, Donnelly P. Inference of population structure using multilocus genotype data. Genetics. 2000;155(2):945-959.4 and https://web.stanford.edu/group/pritchardlab/structure.html are incorporated by reference for all purposes.

The ancestral composition includes one or more ancestral groups. An ancestral group may correspond to an ethnic origin or a group of people descended from one or more common ancestors. The granularity of an ancestral group may vary depending on embodiments and methods used in delineating and combining ancestral groups and subgroups. For example, in some embodiments, the communities may be African, Asian, European, etc. In another embodiment, the European community may be divided into Irish, German, Swedes, etc. In yet another embodiment, the Irish may be further divided into Irish in Ireland and Irish immigrated to America. The ancestral group classification may also depend on whether a population is admixed or unadmixed. For an admixed population, the classification may further be divided based on different ethnic origins in a geographical region.

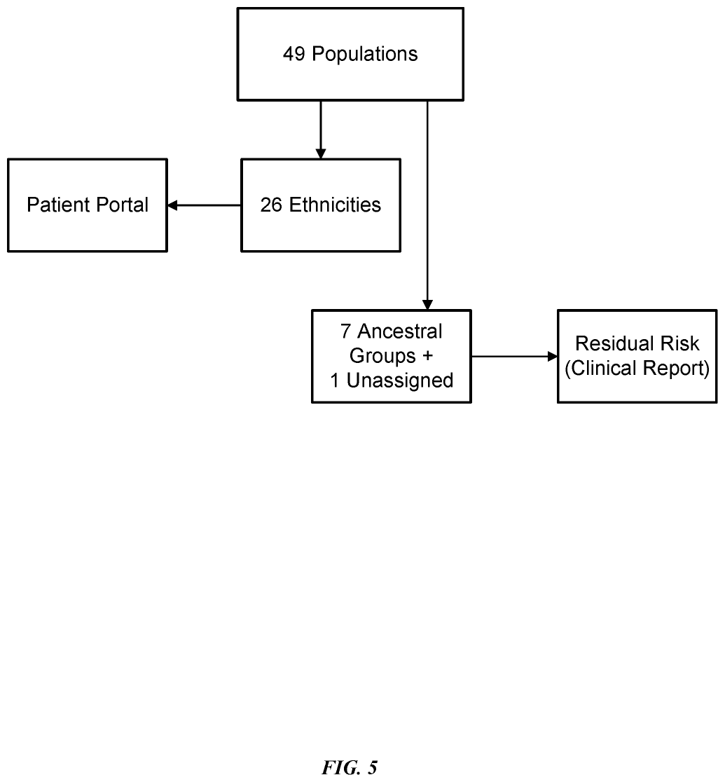

FIG. 5 and Tables 2, 3, and 4 illustrate one example of the classification of ancestral groups that are formed by binning one or more ethnicities into an ancestral group. In this example, each ancestral group is a large group that includes multiple ethnicities. Each ethnicity may be a subset of an ancestral group. The ethnicities are further grouped from different populations. In a patient portal, a computing device may report the ethnicity of the individual while using the larger ancestral group to determine residual risk. The classification shown in FIG. 5 is merely one example of how ancestral groups are defined. In some embodiments, an ancestral group may also correspond to an ethnicity or a population.

By way of example, ancestries are assigned into at least 49 different populations as shown in the Table 2 below. In various embodiments, different population groups can be defined and created.

TABLE 2
49 Populations

ASHKENAZI
BALOCH1-MAKRAN I-
BRAHUI
BANTUKENYA
BANTUNIGERIA
BENGALI
BIAKA
CAFRICA
CAMBODIA-THAI
CRETE
CAMERICA
CYPRUS-MALTA-SICILY
EAFRICA
EASIA
EASTSIBERIA
FINNISH
GAMBIA
GUJARAT
GUJARAT PATEL
HADZA
HAZARA-UYGUR-UZBEK
ITALY
JAPAN-KOREA
KALASH
MENDE
MILAN
NAFRICA
NCASIA
NEAREAST
NEASIA
NEEUROPE
NEUROPE
NGANASAN
NITALY1
NITALY2
NITALY3
OCEANIA
PATHAN-SINDHI-BURUSHO
SAFRICA
SAMERICA
SARDINIA
SBALKANS
SCANDINAVIA
SCOTLAND
SEASIA
SSASIA
SWEUROPE
TAIWAN
TUBALAR
TURK-IRAN-CAUCASUS

The determination of the molecular ancestry of the individual results in two sets of ancestry data as shown in FIG. 3. The first set includes the binning of the populations (e.g., 49 populations) described above into a grouping of different ethnicities (e.g, 26 ethnicities). These ethnicities may be reported to the individual in a patient portal for purposes of identifying their ancestral background. The 26 ethnicities are shown in Table 3 below. In various embodiments, the 49 (or another number of populations) can be binned into other ethnicity subsets than those exemplified in Table 3.:

TABLE 3
26 Ethnicity Subsets

AMERICAS
ASHKENAZI
BENGALI
CAFRICA
CASIA
EAFRICA
EASIA
EMED
FINLAND
INDPAK
NAFRICA
NCASIA
NEAREAST
NEASIA
NEEUROPE
NEUROPE
NITALY
NNEUROPE
OCEANIA
SAFRICA
SCANDINAVIA
SEASIA
SSASIA
SWEUROPE
TURK-IRAN-CAUCASUS
WAFRICA

For the calculation of residual risk, the original grouping of 49 populations is binned into a set of 7 ancestries (Ancestry Codes) as shown in Table 4 below. For genetic variations that are of unknown origin, an eighth category exists to encompass the unassigned populations. In other embodiments, the 49 (or another number of populations) can be binned into other sets of ancestral groups.

TABLE 4

| Ancestry Codes (7 Ancestries) | Grouped Populations |
|---|---|
| AFR | SAFRICA |
| | CAFRICA |
| | BANTUKENYA |
| | MENDE |
| | EAFRICA |
| | HADZA |
| | BIAKA |
| | BANTUNIGERIA |
| | GAMBIA |
| AMR | SAMERICA |
| | CSAMERICA |
| ASJ | ASHKENAZI |
| EAS | NEASIA |
| | NGANASAN |
| | EASTSIBERIA |
| | TAIWAN |
| | EASIA |
| | SEASIA |
| | JAPAN-KOREA |
| | TUBALAR |
| | CAMBODIA- |
| | THAI NCASIA |
| | OCEANIA |
| FIN | FINNISH |
| NFE | SCANDINAVIA |
| | NITALY1 |
| | NITALY2 |
| | NITALY3 |
| | HAZARA-UYGUR-UZBEK |
| | SARDINIA |
| | TURK-IRAN-CAUCASUS |
| | KALASH |
| | PATHAN-SINDHI-BURUSHO |
| | BALOCHI-MAKRANI- |
| | BRAHUINEEUROPE |
| | NEAREAST |
| | NEUROPE |
| | NAFRICA |
| | ITALY |
| | SWEUROPE |
| | SCOTLAND |
| | MILAN |
| | CYPRUS-MALTA-SICILY |
| | CRETE |
| | SBALKANS |
| SAS | SSASIA |
| | BENGALI |
| | GUJARAT PATEL |
| | GUJARAT |

For a particular disease that is tested negative, the computing device retrieves 430 one or more group residual risk values corresponding to one or more ancestral groups in the composition of the individual. Each group residual risk value may be specific to an ancestral group and may be determined based on a carrier frequency and a detection rate specific to the ancestral group. The results of the expanded carrier screening process 300 inform the applicability of residual risk calculations. The residual risk may pertain to pathogenic variants undetected by the expanded carrier screen. For each gene that is determined to be negative for pathogenic variants, ancestry-specific information is obtained from a library pertaining to the carrier frequency and test detection rate. An analytical detection rate is also obtained that is not ancestry specific and is specific to the analytical technique used to detect the presence or lack thereof of a disease.

The group residual risk of a particular disease may be determined from the carrier frequency of the ancestral group and the detection rate of the carrier status in the ancestral group with respect to the disease. The group residual risk value is a statistical value of the residual risk for members in the ancestral group. The determination of the group residual value may be based on a Bayesian relationship among the group residual value, the carrier frequency, and the detection rate. The carrier frequency may correspond to a priori risk of being a carrier of a member in an ancestral group. The detection rate may be an empirical data that represents the rate of disease carriers under the carrier screening that will be detected positive. A sequencing result may detect a large number of variants, but variants that currently are not linked to a genetic disease are often not reported. The variants that are not yet linked or unknown to be pathogenic and other unknown factors result in a detection rate that is lower than 100%. The detection rate based on genetic testing may be unchanged. The carrier frequency and detection rate may provide a more accurate risk assessment when a negative carrier result is obtained.

The computing device assigns 440 metadata to the individual profile. The metadata may include a personalized residual risk of the individual with respect to a genetic disease that is tested negative. The personalized residual risk may be determined based on the one or more group residual risk values of the one or more ancestral groups in the sequence dataset. For example, the personalized residual risk may be determined based on a weighted average of the one or more group residual risk values weighted according to the ancestral composition. The personalized residual risk may also be not weighted. In some embodiments, the personalized residual risk is determined based on the highest weighted residual risk of a particular ancestral group (e.g., Example 2 below).

For genetic screening of a prospective offspring between two prospective parents, the process 400 may be carried out for the first parent and repeated for a second parent. The personalized residual risk of the prospective offspring is determined from a first personalized residual risk corresponding to the first parent and a second personalized residual risk corresponding to the second parent. For the second parent, a second sequence dataset may be retrieved. The ancestral composition corresponding to the second parent may be determined. The residual risk of the second parent may also be determined.

In some embodiments, the process 400 uses low-pass whole genome sequencing technology (LPWGS) to run global ancestry on patient samples to accurately identify the ancestral background of each genetic locus that is on the carrier screen. Using carrier frequencies specific for each ancestral group, the patient will receive a personalized residual risk that considers their ethnic makeup at each locus that has been determined to be negative by carrier screening. By using this approach, each individual's carrier screen will be unique and tailored to return the most accurate results.

The process may also use ancestry inference and genotype imputation software, which are used to complement existing clinical tests by updating risk scores by taking into account underlying ancestry information in the patient. The determination of the ancestral composition may rely on a highly curated reference set of 3.3M+SNPs in various reference populations. Using these methodologies the world-wide reference panel of 49 populations as in Table 2 can be collapsed into 7 continental bins as in Table 4.

In perform ancestry inference, the computing device may set a minimum threshold (e.g., >5%, but another threshold value may also be used) for an ancestral group when determining whether to include an ancestral group in the ancestral composition for an individual. The computing device may use that information to adjust risk scores given results from companion tests on a gene-by-gene basis.

### EXAMPLE COMPUTATIONS

The following examples further describe and demonstrate embodiments. The examples are given solely for the purpose of illustration and are not to be construed as limitations of this disclosure, as many variations thereof are possible without departing from the spirit and scope of the invention.

### EXAMPLE 1

### Calculation of Residual Risk for an Individual being a Carrier of a Disease

An individual tested negative on a carrier screen for a specific disease. Despite the negative result, there exists a residual risk that the individual is a carrier for the disease. The individual was found to have >5% ancestry percentages for AFR, AMR, ASJ, EAS, FIN, and Unassigned Ancestries and therefore all of these ancestries are considered in the assignment of residual risks. The residual risks for each ancestry component were calculated using Bayesian probability using the ancestry-specific carrier frequencies and detection rates.

| Ancestry | Carrier Frequency | Detection Rate | Residual Risk |
|---|---|---|---|
| AFR | 1 in 25 | 94% | 1 in 401 |
| AMR | 1 in 61 | 87% | 1 in 463 |
| ASJ | 1 in 58 | 87% | 1 in 439 |
| EAS | 1 in 94 | 65% | 1 in 267 |
| FIN | 1 in 24 | >95% | 1 in 461 |
| Unassigned | 1 in 45 | 86% | 1 in 315 |
| (Worldwide) | | | |

### EXAMPLE 2

### Residual Risk Assignment by Weighting

An individual was determined to have three ancestry percentages that are larger than 5%. In this example, the main ancestry is NFE (85%) while SAS and Unassigned ancestries are 6%. The remaining 5 ancestries were found to have percentages less than 5% and compose the unaccounted for 3% of the individual's ancestry composition. Because the residual risk is associated with a specific ancestry, there exists a need to report a single residual risk for the individual being a carrier of the disease. This is accomplished by weighting, wherein the residual risk is multiplied by the ancestry percentage to give a weighted RR for each ancestry component. Then, the weighted residual risk values are compared to one another. The largest weighted RR value is chosen to represent the residual risk that the individual is a carrier for the undetected disease. In this example, the highest weighted RR corresponds to the ancestry that has the largest unweighted residual risk.

It can be appreciated that in other examples, the highest weighted residual risk will not necessarily correspond to the ancestry containing the highest residual risk, especially if said ancestry is present in a low percentage.

| | NFE | SAS | Unassigned |
|---|---|---|---|
| Ancestry % | 85% | 6% | 6% |
| Residual Risk (RR) | 1 in 1,200 | 1 in 13,000 | 1 in 2,000 |
| Fraction RR | 0.0008333 | 7.6923 × 10⁵ | 0.0005 |
| Weighted RR | 0.0007083 | 4.6154 × 10⁶ | 0.00003 |

| | |
|---|---|
| Highest Weighted RR | 0.0007083 |

### EXAMPLE 3

### Residual Risk for Offspring of a Reproductive Couple

A prospective mother and father require knowledge of the residual risk that their offspring will exhibit a certain disease despite both of them testing negative as carriers of the disease. The prospective mother has a residual risk of 1 in 450 for the disease and the prospective father has a residual risk of 1 in 40. The residual risk for an offspring of the reproductive couple is calculated using the following formula:

RR (offspring) =RR (prospective mother) x RR (prospective father) x 0.25 In this example, the offspring will have a residual risk of 1/72,000 for exhibiting the disease.

### EXAMPLE 4

### Calculation of Residual Risk for Offspring of a Reproductive Couple when One Prospective Parent is a Carrier of an Autosomal Recessive Disease

A prospective mother was found to be a carrier for one autosomal recessive disease, cystic fibrosis. A prospective father was found to be a carrier for a different autosomal recessive disease, phenylalanine hydroxylase deficiency. As the reproductive couple was not identified to be carriers for the same condition(s), they are considered at a decreased risk for having offspring exhibiting said conditions. The reproductive risk is calculated using the equation below:

Reproductive risk=RR (positive carrier)×RR (partner)×0.25 where RR (positive carrier)=1/1

Their reproductive risk for the condition(s) described can be found in the table below:

| | Prospective | Prospective | |
|---|---|---|---|
| | mother's residual | father's residual | Couple's |
| Condition | carrier risk | carrier risk | reproductive risk |
| Cystic fibrosis | Carrier | 1/424 | 1/1,696 |
| Phenylalanine | 1/818 | Carrier | 1/3,272 |
| hydroxylase deficiency | | | |

### COMPUTING MACHINE ARCHITECTURE

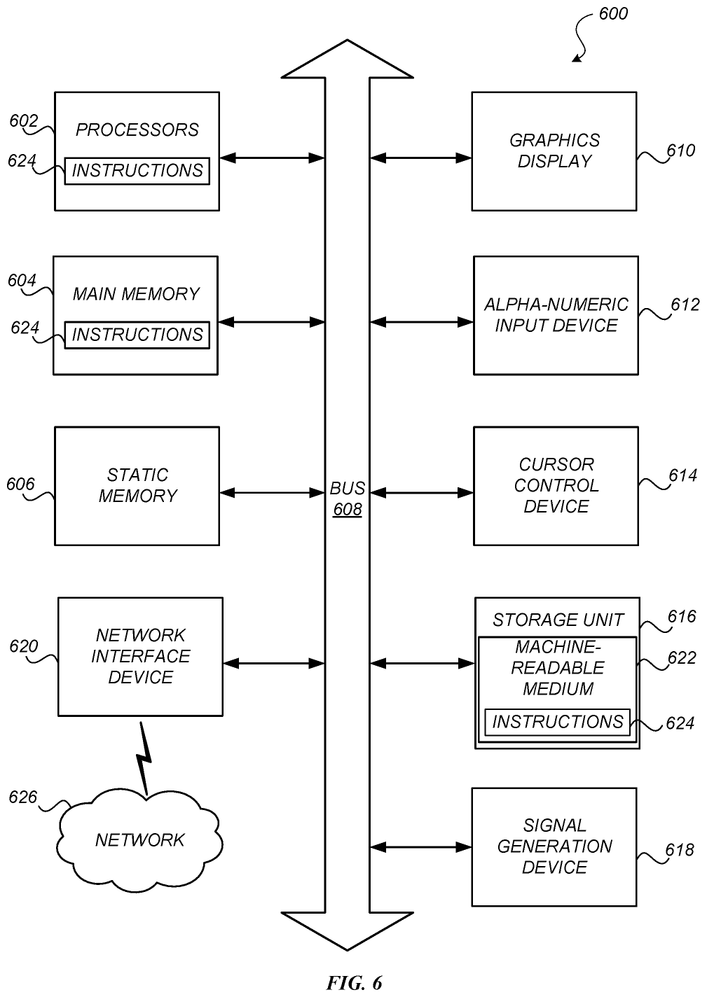

FIG. 6 is a block diagram illustrating components of an example computing machine that is capable of reading instructions from a computer-readable medium and execute them in a processor (or controller). A computer described herein may include a single computing machine shown in FIG. 6, a virtual machine, a distributed computing system that includes multiples nodes of computing machines shown in FIG. 6, or any other suitable arrangement of computing devices.

By way of example, FIG. 6 shows a diagrammatic representation of a computing machine in the example form of a computer system 600 within which instructions 624 (e.g., software, program code, or machine code), which may be stored in a computer-readable medium for causing the machine to perform any one or more of the processes discussed herein may be executed. In some embodiments, the computing machine operates as a standalone device or may be connected (e.g., networked) to other machines. In a networked deployment, the machine may operate in the capacity of a server machine or a client machine in a server-client network environment, or as a peer machine in a peer-to-peer (or distributed) network environment.

The structure of a computing machine described in FIG. 6 may correspond to any software, hardware, or combined components shown in FIG. 1, including but not limited to, the user device 110, the computing server 130, the biomarker data servers 150, and various engines, modules, interfaces, terminals, computing nodes and machines. While FIG. 6 shows various hardware and software elements, each of the components described in FIG. 1 may include additional or fewer elements.

By way of example, a computing machine may be a personal computer (PC), a tablet PC, a set-top box (STB), a personal digital assistant (PDA), a cellular telephone, a smartphone, a web appliance, a network router, an internet of things (IoT) device, a switch or bridge, or any machine capable of executing instructions 624 that specify actions to be taken by that machine. Further, while only a single machine is illustrated, the term “machine” and “computer” may also be taken to include any collection of machines that individually or jointly execute instructions 624 to perform any one or more of the methodologies discussed herein.

The example computer system 600 includes one or more processors 602 such as a CPU (central processing unit), a GPU (graphics processing unit), a TPU (tensor processing unit), a DSP (digital signal processor), a system on a chip (SOC), a controller, a state equipment, an application-specific integrated circuit (ASIC), a field-programmable gate array (FPGA), or any combination of these. Parts of the computing system 600 may also include a memory 604 that store computer code including instructions 624 that may cause the processors 602 to perform certain actions when the instructions are executed, directly or indirectly by the processors 602. Instructions can be any directions, commands, or orders that may be stored in different forms, such as equipment-readable instructions, programming instructions including source code, and other communication signals and orders. Instructions may be used in a general sense and are not limited to machine-readable codes. The processors 602 may include one or more multiply-accumulate units (MAC units) that are used to perform computations of one or more processes described herein.

One and more methods described herein improve the operation speed of the processors 602 and reduces the space required for the memory 604. For example, the various processes described herein reduce the complexity of the computation of the processors 602 by applying one or more novel techniques that simplify the steps in analyzing data and generating results of the processors 602. The algorithms described herein also reduces the size of the models and datasets to reduce the storage space requirement for memory 604.

The performance of certain of the operations may be distributed among the more than processors, not only residing within a single machine, but deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations. Even though in the specification or the claims may refer some processes to be performed by a processor, this should be construed to include a joint operation of multiple distributed processors.

The computer system 600 may include a main memory 604, and a static memory 606, which are configured to communicate with each other via a bus 608. The computer system 600 may further include a graphics display unit 610 (e.g., a plasma display panel (PDP), a liquid crystal display (LCD), a projector, or a cathode ray tube (CRT)). The graphics display unit 610, controlled by the processors 602, displays a graphical user interface (GUI) to display one or more results and data generated by the processes described herein. The computer system 600 may also include alphanumeric input device 612 (e.g., a keyboard), a cursor control device 614 (e.g., a mouse, a trackball, a joystick, a motion sensor, or other pointing instrument), a storage unit 616 (a hard drive, a solid state drive, a hybrid drive, a memory disk, etc.), a signal generation device 618 (e.g., a speaker), and a network interface device 620, which also are configured to communicate via the bus 608.

The storage unit 616 includes a computer-readable medium 622 on which is stored instructions 624 embodying any one or more of the methodologies or functions described herein. The instructions 624 may also reside, completely or at least partially, within the main memory 604 or within the processor 602 (e.g., within a processor's cache memory) during execution thereof by the computer system 600, the main memory 604 and the processor 602 also constituting computer-readable media. The instructions 624 may be transmitted or received over a network 626 via the network interface device 620.

While computer-readable medium 622 is shown in an example embodiment to be a single medium, the term “computer-readable medium” should be taken to include a single medium or multiple media (e.g., a centralized or distributed database, or associated caches and servers) able to store instructions (e.g., instructions 624). The computer-readable medium may include any medium that is capable of storing instructions (e.g., instructions 624) for execution by the processors (e.g., processors 602) and that causes the processors to perform any one or more of the methodologies disclosed herein. The computer-readable medium may include, but not be limited to, data repositories in the form of solid-state memories, optical media, and magnetic media. The computer-readable medium does not include a transitory medium such as a propagating signal or a carrier wave.

In various embodiments, a non-transitory computer readable medium that is configured to store instructions may be used. The instructions, when executed by one or more processors, cause the one or more processors to perform steps described in the above computer-implemented processes or described in any embodiments of this disclosure. In various embodiments, a system may include one or more processors and a storage medium that is configured to store instructions. The instructions, when executed by one or more processors, cause the one or more processors to perform steps described in the above computer-implemented processes or described in any embodiments of this disclosure.

### ADDITIONAL CONSIDERATIONS

Beneficially, various embodiments described herein improve the accuracy and efficiency of existing technologies in the field of sequencing, such as PCR and massively parallel DNA sequencing (e.g., NGS). The embodiments provide solutions to the challenge of generating useful data in a potentially noisy environment introduced by the sequencing and amplification process. A massively parallel DNA sequencing may start with one or more DNA samples, which are randomly cleaved and typically amplified. The parallel nature of massively parallel DNA sequencing results in replicates of nucleotide sequences of each allele. The extent of replication and sequencing at each allele site could vary. Both the amplification process and the sequencing process and the sequencing process have non-trivial error rates. The sequence errors may act to obscure the nucleotide sequences of the true alleles. To reduce the errors, conventionally NGS needs to have certain minimum coverage (e.g., 15-20×) to get the results needed for genetic screening. However, sequencing at such depth may be prohibitively costly for a general genetic screening that tests for hundreds of potential diseases.

Embodiments described reduce the sequencing coverage needed while increasing the accuracy of genetic screening. Embodiments may use a low-pass sequencing that has a low coverage to sample various locations of the genome. Conventionally using NGS that has low coverage is insufficient to determine any carrier risk associated with a genetic disease because the result is too noisy to determine whether the subject is in possession of any pathogenic disease. In some embodiments, the sequence dataset generated by the low-pass sequencing is compared to a reference library of genomes that are associated with different populations. Although the coverage is relatively low (sometimes lower than 0.5×), the sampling is sufficient to generate ancestral group composition with statistically acceptable accuracy. The result of the low-pass sequencing can be used to generate useful information with respect to carrier risk of a large number of diseases. Embodiments described turn potentially data that is conventionally too noisy for carrier screening into useful data that can be used to determine carrier risks for a large number of diseases while allowing a considerably larger (sometimes 20 to 50 folds) number of samples to be sequenced in a single run to due to the low coverage.

The foregoing description of the embodiments has been presented for the purpose of illustration; it is not intended to be exhaustive or to limit the patent rights to the precise forms disclosed. Persons skilled in the relevant art can appreciate that many modifications and variations are possible in light of the above disclosure.

Any feature mentioned in one claim category, e.g. method, can be claimed in another claim category, e.g. computer program product, system, storage medium, as well. The dependencies or references back in the attached claims are chosen for formal reasons only. However, any subject matter resulting from a deliberate reference back to any previous claims (in particular multiple dependencies) can be claimed as well, so that any combination of claims and the features thereof is disclosed and can be claimed regardless of the dependencies chosen in the attached claims. The subject-matter may include not only the combinations of features as set out in the disclosed embodiments but also any other combination of features from different embodiments. Various features mentioned in the different embodiments can be combined with explicit mentioning of such combination or arrangement in an example embodiment or without any explicit mentioning. Furthermore, any of the embodiments and features described or depicted herein may be claimed in a separate claim and/or in any combination with any embodiment or feature described or depicted herein or with any of the features.

Some portions of this description describe the embodiments in terms of algorithms and symbolic representations of operations on information. These operations and algorithmic descriptions, while described functionally, computationally, or logically, are understood to be implemented by computer programs or equivalent electrical circuits, microcode, or the like. Furthermore, it has also proven convenient at times, to refer to these arrangements of operations as engines, without loss of generality. The described operations and their associated engines may be embodied in software, firmware, hardware, or any combinations thereof.

Any of the steps, operations, or processes described herein may be performed or implemented with one or more hardware or software engines, alone or in combination with other devices. In one embodiment, a software engine is implemented with a computer program product comprising a computer-readable medium containing computer program code, which can be executed by a computer processor for performing any or all of the steps, operations, or processes described. The term “steps” does not mandate or imply a particular order. For example, while this disclosure may describe a process that includes multiple steps sequentially with arrows present in a flowchart, the steps in the process do not need to be performed by the specific order claimed or described in the disclosure. Some steps may be performed before others even though the other steps are claimed or described first in this disclosure. Likewise, any use of (i), (ii), (iii), etc., or (a), (b), (c), etc. in the specification or in the claims, unless specified, is used to better enumerate items or steps and also does not mandate a particular order.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein. In addition, the term “each” used in the specification and claims does not imply that every or all elements in a group need to fit the description associated with the term “each.” For example, “each member is associated with element A” does not always imply that all members are associated with an element A. Instead, the term “each” only implies that a member (of some of the members), in a singular form, is associated with an element A. In claims, the use of a singular form of a noun may imply at least one element even though a plural form is not used.

Finally, the language used in the specification has been principally selected for readability and instructional purposes, and it may not have been selected to delineate or circumscribe the patent rights. It is therefore intended that the scope of the patent rights be limited not by this detailed description, but rather by any claims that issue on an application based hereon. Accordingly, the disclosure of the embodiments is intended to be illustrative, but not limiting, of the scope of the patent rights.

What is claimed is:

1. A computer-implemented method, comprising:
retrieving an individual profile for an individual and a sequence dataset associated with the individual profile;
determining an ancestral composition of the sequence dataset, the ancestral composition comprising one or more ancestral groups;
retrieving one or more group residual risk values corresponding to the one or more ancestral groups, each group residual risk value specific to an ancestral group and determined based on a carrier frequency and a detection rate specific to the ancestral group; and
determining a personalized residual risk of the individual being associated with a genetic disease based on the one or more group residual risk values.

2. The computer-implemented method of claim 1, wherein the sequence dataset is a DNA dataset generated by a massively parallel sequencing of a biological sample of the individual.

3. The computer-implemented method of claim 2, wherein the massively parallel sequencing is a low-pass sequencing having a coverage of lower than 5×.

4. The computer-implemented method of claim 2, wherein the massively parallel sequencing is a low-pass sequencing having a coverage of lower than 1×.

5. The computer-implemented method of claim 1, wherein the individual is a prospective parent.

6. The computer-implemented method of claim 1, wherein the individual is a prospective offspring of a first parent and a second parent, and the personalized residual risk of the prospective offspring is determined from a first personalized residual risk corresponding to the first parent and a second personalized residual risk corresponding to the second parent.

7. The computer-implemented method of claim 6, wherein the ancestral composition of the sequence dataset corresponds to a first ancestral composition of the first parent, the sequence dataset corresponds to a first sequence dataset of the first parent, and the computer-implemented method of claim 6 further comprises:
retrieving a second sequence dataset of the second parent; and
determining a second ancestral composition corresponding to the second parent.

8. The computer-implemented method of claim 1, wherein the personalized residual risk is specific to an autosomal recessive or X-linked disease.

9. The computer-implemented method of claim 8, wherein the autosomal recessive or X-linked disease is tested negative by a carrier screening of the individual, and the personalized residual risk corresponds to a risk of the individual being a carrier of the autosomal recessive or X-linked disease despite testing negative in the carrier screening.

10. The computer-implemented method of claim 1, wherein each group residual risk value specific to an ancestral group of the one or more ancestral groups is determined based on a Bayesian relationship among the group residual risk value, the carrier frequency, and the detection rate.

11. The computer-implemented method of claim 1, wherein determining the ancestral composition of the sequence dataset comprises comparing the sequence dataset to a library of ancestry-specific reference sets.

12. The computer-implemented method of claim 1, wherein determining the ancestral composition of the sequence dataset comprises:
determining an ethnicity composition of the sequence dataset, the ethnicity composition comprising one or more ethnicities, an ethnicity being a subset of an ancestral group; and
binning the one or more ethnicities in the ethnicity composition into the ancestral composition.

13. The computer-implemented method of claim 1, wherein the personalized residual risk is determined based on a weighted average of the one or more group residual risk values weighted according to the ancestral composition.

14. The computer-implemented method of claim 1, further comprising:
transmitting the personalized residual risk to an end-user device for display.

15. The computer-implemented method of claim 1, wherein the ancestral composition is a global molecular ancestral composition.

16. The computer-implemented method of claim 1, wherein the ancestral composition is a local molecular ancestral composition.

17. A system comprising:
a computing server comprising a processor and memory, the memory configured to store instructions, the instructions, when executed by the processor, cause the processor to perform a first set of steps comprising:
retrieving an individual profile for an individual and a sequence dataset associated with the individual profile;
determining an ancestral composition of the sequence dataset, the ancestral composition comprising one or more ancestral groups;
retrieving one or more group residual risk values corresponding to the one or more ancestral groups, each group residual risk value specific to an ancestral group and determined based on a carrier frequency and a detection rate specific to the ancestral group; and
determining a personalized residual risk of the individual being associated with a genetic disease based on the one or more group residual risk values; and
a graphical user interface in communication with the computing server, the graphical user interface configured to perform a second set of steps comprising:
receiving the personalized residual risk from the computing server; and
displaying the personalized residual risk.

18. The system of claim 17, wherein the sequence dataset a DNA dataset generated by a massively parallel sequencing of a biological sample of the individual, and the massively parallel sequencing is a low-pass sequencing having a coverage of less than 1×.

19. A method comprising:
receiving one or more biological samples for sequencing;
preparing a first set of nucleic acid samples and a second set of nucleic acid samples from the one or more biological samples;
performing a carrier screening for a genetic disease using the first set of nucleic acid samples, the performing of the carrier screening comprising performing a first sequencing on the first set of nucleic acid samples;
determining that the carrier screening for the genetic disease has a negative result;
performing, responsive to the negative result, a second sequencing on the second set of nucleic acid samples to determine an ancestral composition of the second set of nucleic acid samples; and
determining a personalized residual risk of an individual associated with the genetic disease based on the ancestral composition.

20. The method of claim 19, wherein the first sequencing has a coverage of 10× or higher and the second sequencing has a coverage of 5× or lower.

21. A non-transitory computer readable medium configured to store computer code comprising instructions, the instructions, when executed by one or more processors, cause the one or more processors to perform steps comprising:
retrieving an individual profile for an individual and a sequence dataset associated with the individual profile;
determining an ancestral composition of the sequence dataset, the ancestral composition comprising one or more ancestral groups;
retrieving one or more group residual risk values corresponding to the one or more ancestral groups, each group residual risk value specific to an ancestral group and determined based on a carrier frequency and a detection rate specific to the ancestral group; and
determining a personalized residual risk of the individual being associated with a genetic disease based on the one or more group residual risk values.
